SEQUENCE LISTING (30 sequences)

SEQ ID NO: 1 - protein, 5 aa, Artificial Sequence
  AZD 7789 TIM-3 VH CDR1
SYAMS

SEQ ID NO: 2 - protein, 17 aa, Artificial Sequence
  AZD 7789 TIM-3 VH CDR2
AISGSGGSTYYADSVKG

SEQ ID NO: 3 - protein, 13 aa, Artificial Sequence
  AZD 7789 TIM-3 VH CDR3
GSYGTYYGNYFEY

SEQ ID NO: 4 - protein, 5 aa, Artificial Sequence
  AZD 7789 PD-1 VH CDR1
DYGMH

SEQ ID NO: 5 - protein, 17 aa, Artificial Sequence
  AZD 7789 PD-1 VH CDR2
YISSGSYTIYSADSVKG

SEQ ID NO: 6 - protein, 13 aa, Artificial Sequence
  AZD 7789 PD-1 VH CDR3
RAPNSFYEYYFDY

SEQ ID NO: 7 - protein, 11 aa, Artificial Sequence
  AZD 7789 TIM-3 VL CDR1
GGDNIGGKSVH

SEQ ID NO: 8 - protein, 7 aa, Artificial Sequence
  AZD 7789 TIM-3 VL CDR2
YDSDRPS

SEQ ID NO: 9 - protein, 11 aa, Artificial Sequence
  AZD 7789 TIM-3 VL CDR3
QVLDRRSDHFL

SEQ ID NO: 10 - protein, 18 aa, Artificial Sequence
  AZD 7789 PD-1 VL CDR1
SASSKHTNLYWSRHMYWY

SEQ ID NO: 11 - protein, 7 aa, Artificial Sequence
  AZD 7789 PD-1 VL CDR2
LTSNRAT

SEQ ID NO: 12 - protein, 7 aa, Artificial Sequence
  AZD 7789 PD-1 VL CDR3
QQWSSNP

SEQ ID NO: 13 - protein, 11 aa, Artificial Sequence
  TIM-3 (#62) VL CDR3
QVLDRRSDHWL

SEQ ID NO: 14 - protein, 122 aa, Artificial Sequence
  AZD 7789 TIM-3 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGQGTLVTVSS

SEQ ID NO: 15 - protein, 452 aa, Artificial Sequence
  AZD 7789 TIM-3 HC
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSVDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 108 aa, Artificial Sequence
  TIM3 (#62) Variable Light VL
QTVLTQPPSVSVAPGKTASISCGGDNIGGKSVHWYQQKPGQAPVLVIYYDSDRPSGIPQRFSGSNSGNTATLTIHRVEAGDEADYYCQVLDRRSDHWLFGGGTKLTVL

SEQ ID NO: 17 - protein, 108 aa, Artificial Sequence
  AZD 7789 TIM-3 VL
SYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGGGTKLTVL

SEQ ID NO: 18 - protein, 214 aa, Artificial Sequence
  AZD 7789 TIM-3 LC
SYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGGGTKLTVLGQPKAAPSVTLFPPCSEELQANKATLVCLISDFYPGAVTVAWKADSSPVKAGVETTTPSKQSNNKYAASSYLSLTPEQWKSHRSYSCQVTHEGSTVEKTVAPTEVS

SEQ ID NO: 19 - protein, 122 aa, Artificial Sequence
  AZD 7789 PD-1 VH
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSS

SEQ ID NO: 20 - protein, 452 aa, Artificial Sequence
  AZD 7789 PD-1 HC
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 112 aa, Artificial Sequence
  AZD 7789 PD-1 VL
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIK

SEQ ID NO: 22 - protein, 219 aa, Artificial Sequence
  AZD 7789 PD-1 LC
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 710 aa, Artificial Sequence
  TIM-3 Heavy Chain
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVWDVSHEDPEVKFNWYVDGVEVHNAKTKPREEQYNSTYRWSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGGGSGGGGSEVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARGSYGTYYGNYFEYWGQGTLVTVSSGGGGSGGGGSGGGGSGGGGSSYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGCGTKLTVL

SEQ ID NO: 24 - protein, 218 aa, Artificial Sequence
  TIM-3 Light Chain Variable Region
QIVLTQSPATLSLSPGERATLSCSASSKHTNLYWSRHMYWYQQKPGQAPRLLIYLTSNRATGIPARFSGSGSGTDFTLTISSLEPEDFAVYYCQQWSSNPFTFGQGTKLEIKRTVAAPSVFIFPPSDEQLKSGTASWCLLNNFYPREAKVQWKVDNALQSGNSQESVTEQDSKDSTYSLSSTLTLSKADYEKHKVYACEVTHQGLSSPVTKSFNRGEC

SEQ ID NO: 25 - protein, 720 aa, Artificial Sequence
  TIM-3 Heavy Chain
EVQLVESGGGLVQPGGSLRLSCAASGFTFSDYGMHWVRQAPGKGLEWVAYISSGSYTIYSADSVKGRFTISRDNAKNSLYLQMNSLRAEDTAVYYCARRAPNSFYEYYFDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVWDVSHEDPEVKFNWYVDGVEVHNAKTKPREEQYNSTYRWSVLTVLHQDWLNGKEYKCKVSNKALPASIEKTISKAKGQPREPQVYTLPPSREEMTKNQVSLTCLVKGFYPSDIAVEWESNGGGGSGGGGSEVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARGSYGTYYGNYFEYWGQGTLVTVSSGGGGSGGGGSGGGGSGGGGSSYVLTQPPSVSVAPGKTARITCGGDNIGGKSVHWYQQKPGQAPVLVIYYDSDRPSGIPERFSGSNSGNTATLTISRVEAGDEADYYCQVLDRRSDHFLFGCGTKLTVLGGGGSGGGGSGQPENNYKTTPPVLDSDGSFFLYSKLTVDKSRWQQGNVFSCSVMHEALHNHYTQKSLSLSPGK

SEQ ID NO: 26 - protein, 218 aa, Artificial Sequence; identical to SEQ ID NO: 24
  TIM-3 Light Chain

SEQ ID NO: 27 - protein, 122 aa, Artificial Sequence
  TIM3 (#62) Variable Heavy VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARGSYGTYYGNYFEYWGRGTLVTVSS

SEQ ID NO: 28 - protein, 288 aa, Artificial Sequence
  Amino acid sequence of human PD-1 protein
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 29 - protein, 109 aa, Artificial Sequence
  Human TIM-3 IgV domain
SEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIK

SEQ ID NO: 30 - protein, 301 aa, Artificial Sequence
  Human TIM-3 protein
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

(19) United States
(12) Patent Application Publication
(10) Pub. No.: US 2022/0332818 A1
(43) Pub. Date: Oct. 20, 2022

(54) BISPECIFIC ANTIBODY TARGETING PD-1 AND TIM-3

(71) Applicant: MedImmune, LLC, Gaithersburg, MD (US)

(72) Inventors: Kristen POLLIZZI, Gaithersburg, MD (US); Scott A. HAMMOND, Gaithersburg, MD (US); Yariv MAZOR, Wilmington, DE (US); Trinity PERRY, Gaithersburg, MD (US); Stacy PRYTS, Gaithersburg, MD (US); Ashvin R. JAISWAL, Gaithersburg, MD (US); Vaheh OGANSEYAN, Gaithersburg, MD (US); Chunning YANG, Gaithersburg, MD (US); Raffael KUREK, Cambridge (GB); Natalia CEAICOVSCAIA, Wilmington, DE (US); Charles FERTE, Wilmington, DE (US); Eleanor CLANCY-THOMPSON, Gaithersburg, MD (US)

(21) Appl. No.: 17/718,774

(22) Filed: Apr. 12, 2022

Related U.S. Application Data
(60) Provisional application No. 63/174,156, filed on Apr. 13, 2021.

(51) Int. Cl. C07K 16/28; A61P 35/00

(52) U.S. Cl. CPC C07K 16/2803; A61P 35/00; A61K 2039/505

(57) ABSTRACT

The present disclosure provides methods of altering engagement between T-cell immunoglobulin and mucin domain containing protein-3 (TIM-3) and phosphatidylserine (PS) in a subject. Also provided are methods of treatment using TIM-3 binding protein wherein the TIM-3 binding domain specifically binds to the C′C″ and DE loops of the immunoglobulin variable (IgV) domain of TIM-3.

### 1. FIELD

The present disclosure relates generally to mechanisms of action and methods of treatment using a T-cell immunoglobulin and mucin domain containing protein-3 (TIM-3) binding protein, wherein the TIM-3 binding region specifically binds to the immunoglobulin variable (IgV) domain of TIM-3.

### 2. BACKGROUND

Cancer continues to be a major global health burden. Despite progress in immuno-oncology, there continues to be an unmet medical need for effective therapies, especially for those patients with immuno-oncology (IO) acquired resistance.

A number of molecular targets have been identified for their potential utility as IO therapeutics against cancer. Some molecular targets that are being investigated for their therapeutic potential in the area of immuno-oncology therapy include cytotoxic T lymphocyte antigen-4 (CTLA-4 or CD152), programmed death ligand 1 (PD-L1 or B7-H1 or CD274), Programmed Death-1 (PD-1), OX40 (CD134 or TNFRSF4) and T-cell inhibitory receptor T-cell immunoglobulin and mucin-domain containing-3 (TIM3). However, not all patients respond to immune-therapy and some patients stop responding over time. Reasons for such IO-acquired resistance have eluded researchers.

As such, there remains a need to continue to identify candidate targets for immunotherapies, in particular immunotherapies that overcome IO-acquired resistance and enhance patient response above current clinically evaluated immunotherapeutic strategies.

### 3. SUMMARY

Provided herein are methods of altering engagement between T-cell immunoglobulin and mucin domain containing protein-3 (TIM-3) and phosphatidylserine (PS) in a subject, the method comprising administering to the subject a TIM-3 binding protein comprising a TIM-3 binding domain, wherein the TIM-3 binding domain specifically binds to the C′C″ and DE loops of the immunoglobulin variable (IgV) domain of TIM-3. In some aspects, administration of the TIM-3 binding protein increases anti-tumor activity in a subject relative to no antibody administration. In some aspects, administration of the TIM-3 binding protein increases anti-tumor activity in a subject relative to administration of a TIM-3 binding protein that binds to the PS binding cleft (FG and CC′ loops) of the IgV domain of TIM-3.

Also provided herein are methods of increasing T cell mediated anti-tumor activity in a subject, the method comprising administering to the subject a TIM-3 binding protein comprising TIM-3 binding domain, wherein the TIM-3 binding domain specifically binds to the C′C″ and DE loops of the IgV domain of TIM-3. In some aspects, the T cell mediated anti-tumor activity in the subject is increased relative to no antibody administration. In some aspects, the T cell mediated anti-tumor activity in the subject is increased relative to administration of a TIM-3 binding protein that binds to the PS binding cleft (FG and CC′ loops) of the IgV domain of TIM-3.

In some aspects of the methods disclosed herein, administration of the TIM-3 binding protein increases dendritic cell phagocytosis of apoptotic tumor cells in a subject relative to no antibody administration. In some aspects, administration of the TIM-3 binding protein increases dendritic cell phagocytosis of apoptotic tumor cells in a subject relative to administration of a TIM-3 binding protein that binds to the PS binding cleft (FG and CC′ loops) of the IgV domain of TIM-3.

In some aspects of the methods disclosed herein, administration of the TIM-3 binding protein increases dendritic cell cross-presentation of tumoral antigen in a subject relative to no antibody administration. In some aspects, administration of the TIM-3 binding protein increases dendritic cell cross-presentation of tumoral antigen in a subject relative to administration of a TIM-3 binding protein that binds to the PS binding cleft (FG and CC′ loops) of the IgV domain of TIM-3.

Also provided herein are methods of promoting dendritic cell phagocytosis of tumor cells in a subject, the method comprising administering to the subject a TIM-3 binding protein comprising a TIM-3 binding domain, wherein the TIM-3 binding domain specifically binds to the C′C″ and DE loops of the IgV domain of TIM-3.

Also provided herein are methods of increasing dendritic cell cross-presentation of tumor antigens in a subject, the method comprising administering to the subject a TIM-3 binding protein comprising a TIM-3 binding domain, wherein the TIM-3 binding domain specifically binds to the C′C″ and DE loops of the IgV domain of TIM-3. In some aspects, the level of dendritic cell cross-presentation is increased relative to no antibody administration. In some aspects, the level of dendritic cell cross-presentation is increased relative to administration of a TIM-3 binding protein that binds to the PS binding cleft (FG and CC′ loops) of the IgV domain of TIM-3.

In some aspects of the methods disclosed herein, administration of the TIM-3 binding protein increases IL-2 secretion upon engagement to TIM-3 positive T cells in a subject relative to no antibody administration. In some aspects, administration of the TIM-3 binding protein increases IL-2 secretion upon engagement to TIM-3 positive T cells in a subject relative to administration of a TIM-3 binding protein that binds to the PS binding cleft (FG and CC′ loops) of the IgV domain of TIM-3.

In some aspects of the methods disclosed herein, administration of the TIM-3 binding protein results in inhibition of tumor growth in the subject. In some aspects, the tumor is an advanced or metastatic solid tumor. In some aspects, the subject has one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, lung cancer, esophageal cancer, gastric cancer, biliary tract tumors, urothelial carcinoma, Hodgkin lymphoma, non-hodgkin lymphoma, myelodysplastic syndrome, and acute myeloid leukemia.

In some aspects of the methods disclosed herein, the subject has immune-oncology (IO) acquired resistance.

Also provided herein are methods of treating a cancer in a subject with 10 acquired resistance, wherein the method comprises administering to the subject a TIM-3 binding protein comprising a TIM-3 binding domain, wherein the TIM-3 binding domain specifically binds to the C′ C″ and DE loops of the IgV domain of TIM-3. In some aspects, the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, lung cancer, esophageal cancer, gastric cancer, biliary tract tumors, urothelial carcinoma, Hodgkin lymphoma, non-hodgkin lymphoma, myelodysplastic syndrome, and acute myeloid leukemia. In some aspects, the subject is a human. In some aspects, the subject has documented Stage III, which is not amenable to curative surgery or radiation, or Stage IV non-small cell lung carcinoma (NSCLC). In some aspects, the NSCLC is squamous or non-squamous NSCLC. In some aspects, the subject has a radiologically documented tumor progression or clinical deterioration following initial treatment with an anti-PD-1/PD-L1 therapy for a minimum of 3-6 months, as monotherapy or in combination with chemotherapy, and had signs of initial clinical benefit, i.e. disease stabilization or regression.

In some aspects of the methods disclosed herein, the IO acquired resistance is defined as: (i) Exposure of less than 6 months to anti-PD-1/PD-L1 monotherapy with initial best overall response (BOR) of partial regression or complete regression followed by disease progression during treatment or disease progression less than or equal to 12 weeks after anti-PD-1/PD-L1 treatment discontinuation; or (ii) Exposure of greater than or equal to 6 months to anti-PD-1/PD-L1 therapy alone or in combination with chemotherapy with BOR of disease stabilization, partial regression, or complete regression followed by disease progression during treatment or disease progression less than or equal to 12 weeks after anti-PD-1/PD-L1 treatment discontinuation.

In some aspects of the methods disclosed herein, the IO acquired resistance is defined as exposure of greater than or equal to 6 months to anti-PD-1/PD-L1 therapy alone or in combination with chemotherapy; a best overall response (BOR) of disease stabilization, partial regression, or complete regression followed by disease progression during treatment or disease progression less than or equal to 12 weeks after anti-PD-1/PD-L1 treatment discontinuation. In some aspects, the subject's PD-L1 tumor proportion score (TPS) is greater than or equal to 1%. In some aspects, the subject has not received prior systemic therapy in a first-line setting. In some aspects, the prior systemic therapy is an IO therapy other than an anti-PD-1/PD-L1 therapy. In some aspects, the subject received prior neo/adjuvant therapy but did not progress for at least 12 months following the last administration of an anti-PD-1/PD-L1 therapy. In some aspects, the subject's PD-L1 TPS is greater than or equal to 50%.

In some aspects of the methods disclosed herein, the TIM-3 binding protein comprises Complementarity-Determining Regions (CDRs): HCDR1, HCDR2, HCDR3, LCDR1, LCDR2, and LCDR3 comprising the amino acid sequences of SEQ ID NOs: 1, 2, 3, 7. 8, and 9, respectively, or SEQ ID NOs: 1, 2, 3, 7, 8, and 13, respectively.

In some aspects of the methods disclosed herein, the TIM-3 binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 29).

In some aspects of the methods disclosed herein, the TIM-3 binding protein further comprises a Programmed cell death protein 1 (PD-1) binding domain. In some aspects, the TIM-3 binding domain comprises a first set of Complementarity-Determining Regions (CDRs): HCDR1, HCDR2, HCDR3, LCDR1, LCDR2, and LCDR3 comprising the amino acid sequences of SEQ ID NOs: 1, 2, 3, 7, 8, and 9 or 1, 2, 3, 7, 8, and 13, respectively; and the PD-1 binding domain comprises a second set of CDRs: HCDR1, HCDR2, HCDR3, LCDR1, LCDR2, and LCDR3 comprising the amino acid sequences of SEQ ID NOs: 4, 5, 6, 10, 11, and 12, respectively.

In some aspects, the TIM-3 binding protein comprises a first heavy chain variable domain (VH) comprising the amino acid sequence of SEQ ID NO: 14, a first light chain variable domain (VL) comprising the amino acid sequence of SEQ ID NO: 17, a second heavy chain VH comprising the amino acid sequence of SEQ ID NO: 19, and a second light chain VL comprising the amino acid sequence of SEQ ID NO: 21. In some aspects, the TIM-3 binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22. In some aspects, the TIM-3 binding protein comprises a first heavy chain comprising the amino acid sequence of SEQ ID NO: 23, a first light chain comprising the amino acid sequence of SEQ ID NO: 24, a second heavy chain comprising the amino acid sequence of SEQ ID NO: 23, and a second light chain comprising the amino acid sequence of SEQ ID NO: 24. In some aspects, the TIM-3 binding protein comprises a first heavy chain comprising the amino acid sequence of SEQ ID NO: 25, a first light chain comprising the amino acid sequence of SEQ ID NO: 26, a second heavy chain comprising the amino acid sequence of SEQ ID NO: 25, and a second light chain comprising the amino acid sequence of SEQ ID NO: 26.

In some aspects of the methods disclosed herein, the TIM-3 binding protein comprises an aglycosylated Fc region. In some aspects, the TIM-3 binding protein comprises a deglycosylated Fc region. In some aspects, the TIM-3 binding protein comprises an Fc region which has reduced fucosylation or is afucosylated.

Also disclosed herein are methods of treating NSCLC in a subject having advanced or metastatic NSCLC, the method comprising administering to the subject a bispecific binding protein comprising a PD-1 binding domain and a TIM-3 binding domain, wherein the bispecific binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22, and wherein the subject has 10 acquired resistance.

Also disclosed herein are methods of inhibiting growth of a non-small cell lung tumor in a subject having an advanced or metastatic tumor, the method comprising administering to the subject a bispecific binding protein comprising a PD-1 binding domain and a TIM-3 binding domain, wherein the bispecific binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22, and wherein the subject has 10 acquired resistance. In some aspects, the TIM-3 binding domain specifically binds to the C′C″ and DE loops of the IgV domain of TIM-3. In some aspects, the TIM-3 binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 29). In

In some aspects of the methods disclosed herein, the NSCLC is squamous or non-squamous NSCLC.

### 4. BRIEF DESCRIPTION OF THE FIGURES

FIG. 1A shows that the O13-1 monoclonal antibody (mAb) (the parental anti-TIM-3 mAb to AZD7789) increases binding of human (h-)TIM-3 with phosphatidylserine, as compared to an isotype control, and as compared to an anti-TIM-3 mAb (F9S) which decreases h-TIM-3 interaction with phosphatidylserine (PS).

FIG. 1B shows that monovalent engagement of TIM-3 by AZD7789 is sufficient to increase TIM-3 interaction with phosphatidylserine as compared to bivalent mAb O13-1 binding, and as compared to an isotype control. Error bars represent SEM.

FIG. 2 shows that the O13-1 monoclonal antibody (mAb) and AZD7789 mAb increase binding of human TIM-3 IgV with apoptotic cells, as compared to anti-PD-1 (LO115), a PS-blocking anti-TIM-3 mAb (F9S), Duet LO115/F9S, and E2E which decrease h-TIM-3 interaction with apoptotic cells.

FIG. 3 shows that that AZ anti-TIM3 clones 62GL and O13 mediate a similar effect of enhancing IL-2 production from Jurkat T cells expressing TIM3. Therefore, the one amino acid difference between 62GL and O13 does not affect this phenotype.

FIG. 4 shows a concentration-dependent effect of the anti-TIM-3 mAb O13-1 to drive an increase in IL-2 production of h-TIM-3 expressing Jurkat cells upon T cell stimulation. All other anti-TIM-3 mAbs evaluated demonstrated a concentration dependent decrease in IL-2 production. Error bars represent SEM.

FIG. 5 shows that the observed increase of IL-2 from h-TIM-3 expressing Jurkat cells following stimulation and the addition of anti-TIM-3 mAb O13-1 is ablated when cells are cultured in a high concentration of anti-TIM-3 mAb F9S, which blocks TIM-3 interaction with phosphatidylserine.

FIG. 6 shows that introduction of TIM3 into Jurkat T cells enhances IL-2 production; this is further increased by AZ anti-TIM3 (clone O13) and reduced by competitor-like anti-TIM3 (F9S). This drug effect is dependent on TIM3 binding to phosphatidylserine, as mutation of a residue critical for binding (R111A) abrogates the drug effect, as well as the overall IL-2 production from Jurkat T cells.

FIGS. 7A and 7B show that AZD7789 and its parental anti-TIM-3 mAb O13-1 enhance IFN-γ secretion of stimulated primary human PBMC. FIG. 7A shows IFN-γ secretion of stimulated primary human PBMC as a result of mAb administration in one donor's cells. FIG. 7B shows IFN-γ secretion of stimulated primary human T cells as a result of mAb administration in another donor's cells. The test antibodies are shown in the key. Error bars represent SEM of triplicate wells.

FIGS. 8A and 8B show that AZD7789 can enhance dendritic cell efferocytosis of apoptotic tumor cells. FIG. 8A shows dendritic cell efferocytosis of apoptotic Jurkat cells following administration of the test antibodies or no drug administration in real time (hours). FIG. 8B shows the fold change in efferocytosis following administration of the test antibodies. Fold change in efferocytosis was determined from the no drug treatment group. Error bars represent SEM.

FIGS. 9A and 9B show the percent T cell proliferation from primary human T cells following co-culture with dendritic cells which had been pre-incubated with apoptotic tumor cells in the presence or absence of test antibodies. FIG. 9A shows the percent MART-1 reactive T cell proliferation following co-culture with dendritic cells which had been pre-incubated with apoptotic MART-1 expressing Jurkat cells. FIG. 9B shows the percent CMVppp65 reactive T cell proliferation following co-culture with dendritic cells which had been incubated with apoptotic CMVpp65 expressing Jurkat cells.

FIGS. 10A and 10B show that in a humanized mouse model with adoptive transfer of human tumor reactive T cells, AZD7789 improves tumor control (FIG. 10A) and survival (FIG. 10B) compared to anti-PD-1 alone.

FIGS. 11A and 11B show that treatment with AZD7789 results in decreased tumor growth in a humanized mouse in vivo model as compared to treatment with an anti-PD-1 mAb alone, or in combination with a phosphatidylserine blocking anti-TIM-3 molecule as bivalent mAbs or in a bispecific format. FIG. 11A shows the tumor volume following administration of the test antibodies in a first donor. FIG. 11B shows the tumor volume following administration of the test antibodies in another donor. The horizontal bars represents the intragroup arithmetic mean tumor volume.

FIGS. 12A-12C show that administration of AZD7789 increases IFN-γ secretion of ex vivo stimulated tumor infiltrating lymphocytes taken from mice who progressed on anti-PD-1 treatment. FIG. 12A is a study schematic showing the result on tumor volume of administration with an anti-PD-1 antibody in a humanized mouse model and the ex vivo stimulation of the excised tumor with test drugs. FIG. 12B shows a compilation of fold change in IFN-γ secretion of the ex vivo stimulated tumor infiltrating lymphocytes after addition of anti-PD-1 antibody LO115 and AZD7789, as compared to the isotype control. FIG. 12C shows the increase in IFN-γ secretion of the ex vivo stimulated tumor infiltrating lymphocytes taken from one representative mouse after addition of anti-PD-1 antibody LO115 and AZD7789, as compared to the isotype control.

FIG. 13A is a graph showing the tumor growth curves following treatment with isotype control, AZD7789, anti-PD-1 LO115 antibody alone, and anti-PD-1 followed by sequential treatment of AZD7789, in humanized immunodeficient mice that were subcutaneously engrafted with human PC9-MART-1 tumor cells.

FIGS. 13B and 13C show that sequential treatment with AZD7789 following anti-PD-1 antibody treatment can delay tumor growth in mice as compared to continuous treatment with an anti-PD-1 antibody only. FIG. 13B shows the change in tumor volume following treatment with an isotype control, continuous treatment with the anti-PD-1 antibody LO115, and sequential treatment with AZD7789 following anti-PD-1 antibody treatment. FIG. 13C shows the fold change in tumor volume following continuous treatment with anti-PD-1 antibody LO115 as compared to sequential treatment with AZD7789 following anti-PD-1 antibody treatment.

FIG. 14 is a schematic showing the proposed mechanism of action of AZD7789.

FIG. 15A is a ribbon diagram of human TIM-3 IgV domain bound with Ca++. FIG. 15B is a surface view of human TIM-3 IgV domain bound with Ca++. Strands are labeled with uppercase letters and loops (BC, CC′, C′C″, DE and FG) are highlighted in italics. Phosphatidylserine binds in cleft of domains defined by loops CC′ and FG.

FIGS. 16A and 16B are schematics showing the binding of the AZD7789 and F9S antibodies. FIG. 16A shows binding of F9S near the IgV domain near the CC′ and FG loops, close to the phosphatidylserine and Ca++ ion binding sites. AZD7789 binds the other side of the IgV beta sandwich. FIG. 16B shows the antibody ribbons as bound to the IgV beta sandwich.

### 5. DETAILED DESCRIPTION

In order that the present disclosure may be more readily understood, certain terms are first defined. As used in this application, except as otherwise expressly provided herein, each of the following terms shall have the meaning set forth below. Additional definitions are set forth throughout the application.
5.1 Terminology

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

Where not expressly stated, and unless the context indicates otherwise, the term “antibody” includes monospecific, bispecific, or multi-specific antibodies, as well as a single chain antibody. In some aspects, the antibody is a bispecific antibody. The term “bispecific antibodies” refers to antibodies that bind to two different epitopes. The epitopes can be on the same target antigen or can be on different target antigens.

The term “antibody fragment” refers to a portion of an intact antibody. An “antigen-binding fragment,” “antigen-binding domain,” or “antigen-binding region,” refers to a portion of an intact antibody that binds to an antigen. In the context of a bispecific antibody, an “antigen-binding fragment binds two antigens. An antigen-binding fragment can contain an antigen recognition site of an intact antibody (e.g., complementarity determining regions (CDRs) sufficient to specifically bind antigen). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

A “monoclonal” antibody or antigen-binding fragment thereof refers to a homogeneous antibody or antigen-binding fragment population involved in the highly specific binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

In some aspects, the antibodies or antigen binding fragments thereof disclosed herein are multivalent molecules. The term “valent” as used within the current application denotes the presence of a specified number of binding sites in an antibody molecule. A natural antibody for example or a full length antibody according to the invention has two binding sites and is “bivalent.” The term “tetravalent,” denotes the presence of four binding sites in an antigen binding protein. The term “trivalent” denotes the presence of three binding sites in an antibody molecule. The term “bispecific, tetravalent,” as used herein denotes an antigen binding protein according to the invention that has four antigen-binding sites of which at least one binds to a first antigen and at least one binds to a second antigen or another epitope of the antigen.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that CDRs of the light and heavy chains are primarily responsible for the interaction and specificity of the antibody with antigen. In some aspects of the present disclosure, the variable region is a human variable region. In some aspects of the present disclosure, the variable region comprises rodent or murine CDRs and human framework regions (FRs). In particular aspects of the present disclosure, the variable region is a primate (e.g., non-human primate) variable region. In some aspects of the present disclosure, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat EA et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). In some aspects of the present disclosure, the CDRs of the antibodies described herein have been determined according to the Kabat numbering scheme.

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

As used herein, the term “constant region” and “constant domain” are interchangeable and have their common meanings in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ϵ), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects of the present disclosure, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects of the present disclosure, the light chain is a human light chain.

As used herein, the terms “Programmed Death 1,” “Programmed Cell Death 1,” and “PD-1,” are used interchangeably. The complete PD-1 sequence can be found under NCBI Reference Sequence: NG_012110.1. The amino acid sequence of the human PD-1 protein is:

(SEQ ID NO: 28)
MQIPQAPWPVVWAVLQLGWRPGWFLDSPDRPWNPPTFSPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL.

Programmed Death-1 (“PD-1”) is an approximately 31 kD type I membrane protein member of the extended CD28/CTLA-4 family of T cell regulators (see, Ishida, Y. et al. (1992) Induced Expression Of PD-1, A Novel Member Of The Immunoglobulin Gene Superfamily, Upon Programmed Cell Death,” EMBO J. 11:3887-3895.

PD-1 is expressed on activated T cells, B cells, and monocytes (Agata, Y. et al. (1996) “Expression of the PD-1 Antigen on the Surface of Stimulated Mouse T and B Lymphocytes,” Int. Immunol. 8(5):765-772; Martin-Orozco, N. et al. (2007) “Inhibitory Costimulation and Anti-Tumor Immunity,” Semin. Cancer Biol. 17(4):288-298). PD-1 is a receptor responsible for down-regulation of the immune system following activation by binding of PDL-1 or PDL-2 (Martin-Orozco, N. et al. (2007) “Inhibitory Costimulation and Anti-Tumor Immunity,” Semin. Cancer Biol. 17(4):288-298) and functions as a cell death inducer (Ishida, Y. et al. (1992) “Induced Expression of PD-1, A Novel Member of The Immunoglobulin Gene Superfamily, Upon Programmed Cell Death,” EMBO J. 11: 3887-3895; Subudhi, S. K. et al. (2005) “The Balance of Immune Responses: Costimulation Verse Coinhibition,” J. Molec. Med. 83: 193-202). This process is exploited in many tumors via the over-expression of PD-L1, leading to a suppressed immune response.

PD-1 is a well-validated target for immune mediated therapy in oncology, with positive results from clinical trials in the treatment of melanoma and non-small cell lung cancers (NSCLC), among others. Antagonistic inhibition of the PD-1/PD-L-1 interaction increases T-cell activation, enhancing recognition and elimination of tumour cells by the host immune system. The use of anti-PD-1 antibodies to treat infections and tumors and enhance an adaptive immune response has been proposed (see, U.S. Pat. Nos. 7,521,051; 7,563,869; 7,595,048).

Programmed Death Ligand 1 (PD-L1) is also part of a complex system of receptors and ligands that are involved in controlling T-cell activation. In normal tissue, PD-L1 is expressed on T cells, B cells, dendritic cells, macrophages, mesenchymal stem cells, bone marrow-derived mast cells, as well as various non-hematopoietic cells. Its normal function is to regulate the balance between T-cell activation and tolerance through interaction with its two receptors: programmed death 1 (also known as PD-1 or CD279) and CD80 (also known as B7-1 or B7.1). PD-L1 is also expressed by tumors and acts at multiple sites to help tumors evade detection and elimination by the host immune system. PD-L1 is expressed in a broad range of cancers with a high frequency. In some cancers, expression of PD-L1 has been associated with reduced survival and unfavorable prognosis. Antibodies that block the interaction between PD-L1 and its receptors are able to relieve PD-L1-dependent immunosuppressive effects and enhance the cytotoxic activity of antitumor T cells in vitro. Durvalumab is a human monoclonal antibody directed against human PD-L1 that is capable of blocking the binding of PD-L1 to both the PD-1 and CD80 receptors. The use of anti-PD-L1 antibodies to treat infections and tumors and enhance an adaptive immune response has been proposed (see, U.S. Pat. Nos. 8,779,108 and 9,493,565 incorporated herein by reference in their entirety).

As used herein, the terms “T-cell immunoglobulin and mucin domain containing protein-3” and “TIM-3” are used interchangeably, and include variants, isoforms, species homologs of human TIM-3. TIM-3 is a Type I cell-surface glycoprotein that comprises an N-terminal immunoglobulin (Ig)-like domain, a mucin domain with O-linked glycosylations and with N-linked glycosylations close to the membrane, a single transmembrane domain, and a cytoplasmic region with tyrosine phosphorylation motif(s). TIM-3 is a member of the T cell/transmembrane, immunoglobulin, and mucin (TIM) gene family. The amino acid sequence of the IgV domain of human TIM-3 is:

(SEQ ID NO: 29)
SEVEYRAEVGQNAYLPCFYTPAAPGNLVPVCWGKGACPVFECGNVVLRT
DERDVNYWTSRYWLNGDFRKGDVSLTIENVTLADSGIYCCRIQIPGIMN
DEKFNLKLVIK.

The amino acid sequence of the human TIM-3 protein, including the signal peptide, is:

(SEQ ID NO: 30)
MFSHLPFDCVLLLLLLLLTRSSEVEYRAEVGQNAYLPCFYTPAAPGNLV
PVCWGKGACPVFECGNVVLRTDERDVNYWTSRYWLNGDFRKGDVSLTIE
NVTLADSGIYCCRIQIPGIMNDEKFNLKLVIKPAKVTPAPTRQRDFTAA
FPRMLTTRGHGPAETQTLGSLPDINLTQISTLANELRDSRLANDLRDSG
ATIRIGIYIGAGICAGLALALIFGALIFKWYSHSKEKIQNLSLISLANL
PPSGLANAVAEGIRSEENIYTIEENVYEVEEPNEYYCYVSSRQQPSQPL
GCRFAMP.

The T-cell inhibitory receptor TIM-3 (T-cell immunoglobulin and mucin-domain containing-3) plays a role in regulating antitumor immunity as it is expressed on IFN-gamma producing CD4+ helper 1 (Th1) and CD8+ T cytotoxic1 (Tc1) T cells. It was initially identified as a T-cell inhibitory receptor, acting as an immune checkpoint receptor that functions specifically to limit the duration and magnitude of Th1 and Tc1 T-cell responses. Further research has identified that the TIM-3 pathway may cooperate with the PD-1 pathway to promote the development of a severe dysfunctional phenotype in CD8+ T cells in cancer. It has also been expressed in regulatory T cells (T_{reg}) in certain cancers. TIM-3 is also expressed on cells of the innate immune system including mouse mast cells, subpopulations of macrophages and dendritic cells (DCs), NK and NKT cells, and human monocytes, and on murine primary bronchial epithelial cell lines. TIM-3 can generate an inhibitory signal resulting in apoptosis of Th1 and Tc1 cells, and can mediate phagocytosis of apoptotic cells and cross-presentation of antigen.

The crystal structure of the IgV domain of TIM-3 shows the presence of two anti-parallel β sheets, which are tethered by a disulfide bond. Two additional disulfide bonds formed by four non-canonical cysteines stabilize the IgV domain and reorient a CC′ loop toward a FG loop thereby forming a “cleft” structure that is thought to be involved in ligand binding, and is not found in other IgSF members. Instead, this “cleft” assembly is the signature structure that is identified in all TIM family proteins including TIM-1 and TIM-4. The engagement of the IgV domain by appropriate ligands has been found to be important for the immune-modulatory role of TIM-3, and instrumental for induction of peripheral tolerance and suppression of anti-tumor immunity. The C′C″ loop of TIM-3 involves amino acids after beta strand C′ and before beta strand C″, for example, from amino acids 50 to 54. The DE loop consists of amino acids from 64 to 73, while the CC′ loop and FG loop comprise amino acids 35 to 43 and 92 to 99, respectively.

TIM-3 has several known ligands, such as galectin-9, phosphatidylserine, CEACAM1 and HMGB1. Galectin-9 is an S-type lectin with two distinct carbohydrate recognition domains joined by a long flexible linker, and has an enhanced affinity for larger poly-N-acetyllactosamine-containing structures. Galectin-9 does not have a signal sequence and is localized in the cytoplasm. However, it can be secreted and exerts its function by binding to glycoproteins on the target cell surface via their carbohydrate chains (Freeman G J et al., Immunol Rev. 2010 Can; 235(1): 172-89). Both human and mouse TIM-3 have been shown to be receptors for phosphatidylserine, based on binding studies, mutagenesis, and a co-crystal structure, and it has been shown that TIM-3-expressing cells bound and/or engulfed apoptotic cells expressing phosphatidylserine. Interaction of TIM-3 with phosphatidylserine does not exclude an interaction with galectin-9 as the binding sites have been found to be on opposite sides of the IgV domain.

In view of the involvement the TIM-3 pathway in key immune cell populations that are immunosuppressed in some cancers, it represents an attractive candidate for immuno-oncology therapy. See, Anderson, A. C., Cancer Immunol Res., (2014) 2:393-398; and Ferris, R. L., et al., J Immunol. (2014) 193:1525-1530.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementary determining region (CDR) are replaced by residues from the CDR of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, certain Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the non-human CDR residues to refine and optimize antibody or antigen-binding fragment thereof specificity, affinity, and/or capability. In general, the humanized antibody or antigen-binding fragment thereof will comprise variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects of the present disclosure, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (KD). Affinity can be measured and/or expressed in a number of ways known in the art, including, but not limited to, equilibrium dissociation constant (KD), and equilibrium association constant (KA). The KD is calculated from the quotient of k_{off}/kₒₙ, whereas KA is calculated from the quotient of k_{off}/kₒₙ. Kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore® or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects of the present disclosure, the epitope to which an antibody or antigen-binding fragment thereof specifically binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization can be accomplished using any of the known methods in the art (e.g., Giege R et al., (1994) Acta Crystallogr D Biol Crystallogr 50(Pt 4): 339-350; McPherson A (1990) Eur J Biochem 189: 1-23; Chayen NE (1997) Structure 5: 1269-1274; McPherson A (1976) J Biol Chem 251: 6300-6303). Antibody/antigen-binding fragment thereof:antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.,; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

An antibody that “binds to the same epitope” as a reference antibody refers to an antibody that binds to the same amino acid residues as the reference antibody. The ability of an antibody to bind to the same epitope as a reference antibody can determined by a hydrogen/deuterium exchange assay (see Coales et al. Rapid Commun. Mass Spectrom. 2009; 23: 639-647) or x-ray crystallography.

An antibody is said to “competitively inhibit” or “cross compete” with binding of a reference antibody to a given epitope if it preferentially binds to that epitope or an overlapping epitope to the extent that it blocks, to some degree, binding of the reference antibody to the epitope. Competitive inhibition can be determined by any method known in the art, for example, competition ELISA assays. An antibody can be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects of the present disclosure, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this disclosure are based upon antibodies, in some aspects of the present disclosure, the polypeptides can occur as single chains or associated chains.

As used herein, the term “AZD7789” refers to an anti-TIM-3/PD-1 bispecific antibody that comprises first heavy chain comprising the amino acid sequence of SEQ ID NO: 15, a first light chain comprising the amino acid sequence of SEQ ID NO: 18 and a second heavy chain comprising the amino acid sequence of SEQ ID NO: 20, and a second light chain comprising the amino acid sequence of SEQ ID NO: 22. AZD7789 is disclosed in U.S. Pat. No. 10,457,732, which is herein incorporated by reference in its entirety. The sequences of monoclonal antibody O13-1 and clone 62, discussed herein, are also disclosed in U.S. Pat. No. 10,457,732, which is herein incorporated by reference in its entirety.

As used herein, the term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer,” “administering,” “administration,” and the like, as used herein, refer to methods that can be used to enable delivery of a drug, e.g., an anti-TIM-3/PD-1 binding protein (e.g., antibody or antigen-binding fragment thereof) to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “combination” or “administered in combination” means that an antibody or antigen binding fragment thereof described herein can be administered with one or more additional therapeutic agents. In some aspects, an antibody or antigen binding fragment thereof can be administered with one or more additional therapeutic agents either simultaneously or sequentially. In some aspects, an antibody or antigen binding fragment thereof described herein can be administered with one or more additional therapeutic agent in the same or in different compositions.

As used herein, the terms “subject” and “patient” are used interchangeably. The subject can be an animal. In some aspects of the present disclosure, the subject is a mammal such as a non-human animal (e.g., cow, pig, horse, cat, dog, rat, mouse, monkey or other primate, etc.). In some aspects of the present disclosure, the subject is a cynomolgus monkey. In some aspects of the present disclosure, the subject is a human.

The term “therapeutically effective amount” refers to an amount of a drug, e.g., an anti-TIM-3/PD-lantibody or antigen-binding fragment thereof, effective to treat a disease or disorder in a subject. Terms such as “treating,” “treatment,” “to treat,” “alleviating,” and “to alleviate” refer to therapeutic measures that cure, slow down, lessen symptoms of, and/or halt progression of a pathologic condition or disorder. Thus, those in need of treatment include those already diagnosed with or suspected of having the disorder.

As used in the present disclosure and claims, the singular forms “a,” “an,” and “the” include plural forms unless the context clearly dictates otherwise.

It is understood that wherever aspects of the present disclosure are described herein with the language “comprising,” otherwise analogous aspects described in terms of “consisting of” and/or “consisting essentially of” are also provided.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of 5% to 10% above and 5% to 10% below the value or range remain within the intended meaning of the recited value or range.

Any compositions or methods provided herein can be combined with one or more of any of the other compositions and methods provided herein.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.
5.2 Methods of the Disclosure

In some aspects, the present disclosure provides treatment methods of the novel anti-cancer drug AZD7789, which simultaneously targets PD-1 and TIM-3. In some aspects, the present disclosure provides methods of using AZD7789 in patients with IO-acquired resistance.

X-ray diffraction crystallography studies revealed that the TIM-3 arm of AZD7789 differs from other clinical anti-TIM-3 agents (e.g., monoclonal antibodies) because the TIM-3 arm binds to a unique epitope on the immunoglobulin variable (IgV) extracellular domain of TIM-3. This epitope is outside of the phosphatidylserine binding (FG-CC′ loop) cleft and is comprised of amino acids N12(H-bond), L47, R52(salt bridge), D53(H-bond), V54, N55, Y56, W57, W62, L63)H-bond), N64(H-bond), G65, D66(H-bond), F67, R68(H-bond, salt bridge), K69(H-bond, salt bridge), D71, T75, E77(H-bond). The paratope from the light chain includes residues 28 to 31 of CDR1, 48 to 53 of CDR2 and residue 92 of CDR3. The paratope from the heavy chain includes residues 30 to 33 of CDR1, 52 to 57 of CDR2 and 100 to 108 of CDR3.

The TIM-3 binding arm of AZD7789 binds to the IgV domain at the site opposite from phosphatidylserine binding, and is not directly involved in interaction with residues from those loops. Thus, AZD7789 does not block the interaction of TIM-3 with phosphatidylserine. Instead, AZD7789 increases engagement between TIM-3 and phosphatidylserine. This unique mechanism improves T cell mediated anti-tumor responses over those observed from phosphatidyl serine blocking anti-TIM3 mAbs. Accordingly, in some aspects, the present disclosure provides a method of altering engagement between T-cell immunoglobulin and mucin domain containing protein-3 (TIM-3) and phosphatidylserine (PS) in a subject, the method comprising administering to the subject a TIM-3 binding protein comprising a TIM-3 binding domain, wherein the TIM-3 binding domain specifically binds to the C′ C″ and DE loops of the IgV domain of TIM-3.

### A. Methods of Altering Engagement Between TIM-3 and PS

In some aspects, the disclosure provides a method of altering engagement between T-cell immunoglobulin and mucin domain containing protein-3 (TIM-3) and phosphatidylserine (PS) in a subject. In some aspects, the method comprises administering to the subject the TIM-3 binding protein comprising a TIM-3 binding domain disclosed herein. In some aspects, the TIM-3 binding domain specifically binds to the C′C″ and DE loops of the immunoglobulin variable (IgV) domain of TIM-3.

In some aspects of the method of altering engagement between TIM-3 and PS in a subject disclosed herein, administration of the TIM-3 binding protein increases anti-tumor activity in a subject. In some aspects, anti-tumor activity is increased relative to no binding protein (e.g., antibody) administration. In some aspects, administration of the TIM-3 binding protein increases anti-tumor activity in a subject relative to administration of a TIM-3 binding protein that binds to the PS binding cleft (FG and CC′ loops) of the IgV domain of TIM-3.

In some aspects, the disclosure provides a method of increasing T cell mediated anti-tumor activity in a subject. In some aspects, the method of increasing T cell mediated anti-tumor activity in a subject comprises administering to the subject the TIM-3 binding protein comprising a TIM-3 binding domain disclosed herein. In some aspects, the TIM-3 binding domain specifically binds to the C′C″ and DE loops of the IgV domain of TIM-3.

In some aspects of the method of increasing T cell mediated anti-tumor activity in a subject disclosed herein, the T cell mediated anti-tumor activity in the subject is increased relative to no binding protein (e.g., antibody) administration. In some aspects, the T cell mediated anti-tumor activity in the subject is increased relative to administration of a TIM-3 binding protein that binds to the PS binding cleft (FG and CC′ loops) of the IgV domain of TIM-3.

### B. Methods of Increasing Dendritic Cell Efferocytosis and Cross-Presentation of Tumor Antigens

In some aspects, the disclosure provides a method of increasing dendritic cell phagocytosis of apoptotic tumor cells. In some aspects, administration of the TIM-3 binding protein described herein increases dendritic cell efferocytosis of apoptotic tumor cells. In some aspects, the dendritic cell efferocytosis of apoptotic tumor cells is increased in a subject relative to no binding protein (e.g., antibody) administration. In some aspects, administration of the TIM-3 binding protein increases dendritic cell efferocytosis of apoptotic tumor cells in a subject relative to administration of a TIM-3 binding protein that binds to the PS binding cleft (FG and CC′ loops) of the IgV domain of TIM-3.

In some aspects, the disclosure provides a method of increasing dendritic cell cross-presentation of tumoral antigen in a subject. Cross-presentation is the ability of certain antigen-presenting cells, such as dendritic cells, to take up, process and present extracellular antigens with MHC class I molecules to CD8+ T cells. Cross-priming, the result of this process, is the stimulation of naive cytotoxic CD8+ T cells into activated cytotoxic CD8+ T T cells. This process is necessary for immunity against most tumors and viruses that do not readily infect antigen-presenting cells, but rather tumors and viruses that infect peripheral tissue cells. Cross-presentation is of particular importance, because it permits the presentation of exogenous antigens, which are normally presented by MHC II on the surface of dendritic cells, to also be presented through the MHC I pathway.

In some aspects, administration of the TIM-3 binding protein described herein increases dendritic cell cross-presentation of tumoral antigen in a subject. In some aspects, dendritic cell cross-presentation of tumoral antigen is increased relative to no binding protein (e.g., antibody) administration. In some aspects, administration of the TIM-3 binding protein increases dendritic cell cross-presentation of tumoral antigen in a subject relative to administration of a TIM-3 binding protein that binds to the PS binding cleft (FG and CC′ loops) of the IgV domain of TIM-3.

In some aspects, the disclosure provides a method of promoting dendritic cell efferocytosis of tumor cells in a subject. In some aspects of the method of promoting dendritic cell efferocytosis of tumor cells in a subject, the method comprises administering to the subject the TIM-3 binding protein comprising a TIM-3 binding domain described herein. In some aspects, the TIM-3 binding protein specifically binds to the C′C″ and DE loops of the IgV domain of TIM-3.

In some aspects, the disclosure provides a method of increasing dendritic cell cross-presentation of tumor antigens in a subject. In some aspects, the method of increasing dendritic cell cross-presentation of tumor antigens in a subject comprises administering to the subject the TIM-3 binding protein comprising a TIM-3 binding domain described herein. In some aspects, the TIM-3 binding protein specifically binds to the C′C″ and DE loops of the IgV domain of TIM-3. In some aspects, the level of dendritic cell cross-presentation is increased relative to no binding protein (e.g., antibody) administration. In some aspects, the level of dendritic cell cross-presentation is increased relative to administration of a TIM-3 binding protein that binds to the PS binding cleft (FG and CC′ loops) of the IgV domain of TIM-3.

In some aspects of the methods disclosed herein, administration of the TIM-3 binding protein described herein increases IL-2 secretion upon engagement to TIM-3 positive T cells in a subject. In some aspects, IL-2 secretion is increased relative to no binding protein (e.g., antibody) administration. In some aspects, administration of the TIM-3 binding protein increases IL-2 secretion upon engagement to TIM-3 positive T cells in a subject relative to administration of a TIM-3 binding protein that binds to the PS binding cleft (FG and CC′ loops) of the IgV domain of TIM-3.
5.3 Patient Populations

Provided herein are methods for treating cancers (e.g., squamous or non-squamous NSCLC) in human patients using any method disclosed herein, for example, a bispecific antibody (for example, AZD7789) or antigen-binding fragments thereof. In some aspects, the patient has a solid tumor. In some aspects, the patient has an advanced or metastatic solid tumor.

In some aspects, the subject has one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, lung cancer, esophageal cancer, gastric cancer, biliary tract tumors, urothelial carcinoma, Hodgkin lymphoma, non-hodgkin lymphoma, myelodysplastic syndrome, and acute myeloid leukemia.

Also provided herein are methods for treating cancers in a subject with immune-oncology (IO) acquired resistance. In some aspects, the subject is a human.

In some aspects, the subject has documented Stage III cancer which is not amenable to curative surgery or radiation. In some aspects, the subject has Stage IV non-small cell lung carcinoma (NSCLC). In some aspects, the NSCLC is squamous or non-squamous NSCLC.

In some aspects, the subject with immune-oncology (IO) acquired resistance has a radiologically documented tumor progression or clinical deterioration following initial treatment with an anti-PD-1/PD-L1 therapy for a minimum of 3-6 months, as monotherapy or in combination with chemotherapy, and had signs of initial clinical benefit, i.e. disease stabilization or regression.

In some aspects, the anti-PD-1 therapy is an antibody selected from nivolumab (also known as OPDIVO®, 5C4, BMS-936558, MDX-1106, and ONO-4538), pembrolizumab (Merck; also known as KEYTRUDA®, lambrolizumab, and MK-3475; see WO2008/156712), PDR001 (Novartis; see WO 2015/112900), MEDI-0680 (AstraZeneca; also known as AMP-514; see WO 2012/145493), cemiplimab (Regeneron; also known as REGN-2810; see WO 2015/112800), JS001 (TAIZHOU JUNSHI PHARMA; see Si-Yang Liu et al., J. Hematol. Oncol. 70:136 (2017)), BGB-A317 (Beigene; see WO 2015/35606 and US 2015/0079109), INCSHR1210 (Jiangsu Hengrui Medicine; also known as SHR-1210; see WO 2015/085847; Si-Yang Liu et al, J Hematol. Oncol. 70: 136 (2017)), TSR-042 (Tesaro Biopharmaceutical; also known as ANB011; see WO2014/179664), Pidilizumab (Medivation/CureTech; see U.S. Pat. No. 8,686,119 B2 or WO 2013/014668 A1); GLS-010 (Wuxi/Harbin Gloria Pharmaceuticals; also known as WBP3055; see Si-Yang Liu et al, J Hematol. Oncol. 70: 136 (2017)), AM- 0001 (Armo), STI-1110 (Sorrento Therapeutics; see WO 2014/194302), AGEN2034 (Agenus; see WO 2017/040790), MGA012 (Macrogenics, see WO 2017/19846), and IBI308 (Innovent; see WO 2017/024465, WO 2017/025016, WO 2017/132825, and WO 2017/133540). In some aspects the anti-PD-1 therapy is the PD-1 antagonist AMP-224, which is a recombinant fusion protein comprised of the extracellular domain of the PD-1 ligand programmed cell death ligand 2 (PD-L2) and the Fc region of human IgG. AMP-224 is discussed in U.S. Publ. No. 2013/0017199. The contents of each of these references are incorporated by reference herein in their entirety.

In some aspects, the anti-PD-L1 therapy is an antibody selected from BMS-936559 (also known as 12A4, MDX-1105; see, e.g., U.S. Pat. No. 7,943,743 and WO 2013/173223), atezolizumab (Roche; also known as TECENTRIQ®; MPDL3280A, RG7446; see U.S. Pat. No. 8,217,149; see, also, Herbst et al. (2013) J Clin Oncol 3 1(suppl):3000), durvalumab (AstraZeneca; also known as IMFINZI™, MEDI-4736; see WO 2011/066389), avelumab (Pfizer; also known as BAVENCIO®, MSB-0010718C; see WO 2013/079174), STI-1014 (Sorrento; see WO2013/181634), CX-072 (Cytomx; see WO2016/149201), KN035 (3D Med/Alphamab; see Zhang et al., Cell Discov. 7:3 (March 2017), LY3300054 (Eli Lilly Co.; see, e.g., WO 2017/034916), and CK-301 (Checkpoint Therapeutics; see Gorelik et al., AACR:Abstract 4606 (April 2016)), The contents of each of these references are incorporated by reference herein in their entirety.

In certain aspects of the methods disclosed herein, IO acquired resistance is defined as:
(i) Exposure of less than 6 months to anti-PD-1/PD-L1 monotherapy with initial best overall response (BOR) of partial regression or complete regression followed by disease progression during treatment or disease progression less than or equal to 12 weeks after anti-PD-1/PD-L1 treatment discontinuation; or
(ii) Exposure of greater than or equal to 6 months to anti-PD-1/PD-L1 therapy alone or in combination with chemotherapy with BOR of disease stabilization, partial regression, or complete regression followed by disease progression during treatment or disease progression less than or equal to 12 weeks after anti-PD-1/PD-L1 treatment discontinuation.

In certain aspects of the methods disclosed herein, the IO acquired resistance is defined as exposure of greater than or equal to 6 months to anti-PD-1/PD-L1 therapy alone or in combination with chemotherapy; a best overall response (BOR) of disease stabilization, partial regression, or complete regression followed by disease progression during treatment or disease progression less than or equal to 12 weeks after anti-PD-1/PD-L1 treatment discontinuation.

In some aspects of the methods disclosed herein, the subject's PD-L1 tumor proportion score (TPS) is greater than or equal to 1%. In some aspects, the subject has not received prior systemic therapy in a first-line setting. In some aspects, the prior systemic therapy is an IO therapy other than an anti-PD-1/PD-L1 therapy. In some aspects, the subject received prior neo/adjuvant therapy but did not progress for at least 12 months following the last administration of an anti-PD-1/PD-L1 therapy. In some aspects, the subject's PD-L1 TPS is greater than or equal to 50%.
5.4 Outcomes

A patient treated according to the methods disclosed herein preferably experience improvement in at least one sign of cancer. In one aspect, improvement is measured by a reduction in the quantity and/or size of measurable tumor lesions. In another aspect, lesions can be measured on chest x-rays or CT or MRI films. In another aspect, cytology or histology can be used to evaluate responsiveness to a therapy. In some aspects, tumor response to the administration of the bispecific antibody or antigen-binding fragment thereof can be determined by Investigator review of tumor assessments and defined by the RECIST v1.1 guidelines. Additional tumor measurements can be performed at the discretion of the Investigator or according to institutional practice.

In some aspects, the patient treated exhibits a complete response (CR), i.e., the disappearance of all target lesions. In some aspects, the patient treated exhibits a partial response (PR), i.e., at least a 30% decrease in the sum of the diameters of target lesions, taking as reference the baseline sum diameters. In some aspects, the patient treated exhibits progressive disease (PD), i.e., at least a 20% increase in the sum of diameters of target lesions, taking as reference the smallest sum on study (this includes the baseline sum if that is the smallest on study). In addition to the relative increase of 20%, the sum must also demonstrate an absolute increase of at least 5 mm. (Note: The appearance of one or more new lesions may be considered progression). In some aspects, the patient treated exhibits stable disease (SD), i.e., neither sufficient shrinkage to qualify for PR nor sufficient increase to qualify for PD, taking as reference the smallest sum of diameters while on study.

In another aspect, the patient treated experiences tumor shrinkage and/or decrease in growth rate, i.e., suppression of tumor growth. In some aspects, unwanted cell proliferation is reduced or inhibited. In some aspects, one or more of the following can occur: the number of cancer cells can be reduced; tumor size can be reduced; cancer cell infiltration into peripheral organs can be inhibited, retarded, slowed, or stopped; tumor metastasis can be slowed or inhibited; tumor growth can be inhibited; recurrence of tumor can be prevented or delayed; one or more of the symptoms associated with cancer can be relieved to some extent.

In other aspects, administration of a bispecific antibody or antigen-binding fragment thereof according to any of the methods provided herein produces at least one therapeutic effect selected from the group consisting of reduction in size of a tumor, reduction in number of metastatic lesions appearing over time, complete remission, partial remission, or stable disease.

In some aspects, one or more tumor biopsies can be used to determine tumor response to administration of a bispecific antibody or antigen-binding fragment thereof according to any of the methods provided herein. In some aspects, the sample is a formalin-fixed paraffin embedded (FFPE) sample. In some aspects, the sample is a fresh sample. Tumor samples (e.g., biopsies) can be used to identify predictive and/or pharmacodynamic biomarkers associated with immune and tumor microenvironment. Such biomarkers can be determined from assays including IHC, tumor mutation analysis, RNA analysis, and proteomic analyses. In certain aspects, expression of tumor biomarkers are detected by RT-PCR, in situ hybridization, RNase protection, RT-PCR-based assay, immunohistochemistry,enzyme linked immuosorbent assay, in vivo imaging, or flow cytometry.
5.5 Bispecific Antibodies and Antigen-Binding Fragments Thereof

Provided herein are methods of treating cancers in a subject (e.g., a human subject) comprising administering to the subject antibodies and antigen-binding fragments thereof that specifically bind to TIM-3 and PD-1 (e.g., human TIM-3 and PD-1). In some aspects, TIM-3 and PD-1, (e.g., human TIM-3 and PD-1) antibodies and antigen-binding fragments thereof that can be used in the methods provided herein include AZD7789, a monovalent bispecific humanized immunoglobulin G1 (IgG1) monoclonal antibody (mAb) that specifically binds TIM-3 and PD-1, and targets a unique TIM-3 epitope.

AZD7789 was constructed on the backbone of the DuetMab molecule. The DuetMab design is described in Mazor et al., MAbs. 7(2): 377-389, (2015 Mar.-Apr. 2015), which is hereby incorporated by reference in its entirety. The “DuetMab,” design includes knobs-into-holes (KIH) technology for heterodimerization of 2 distinct heavy chains and increases the efficiency of cognate heavy and light chain pairing by replacing the native disulfide bond in one of the CH1-CL interfaces with an engineered disulfide bond.

AZD7789 includes a knob mutation in the heavy chain comprising a variable region that binds to TIM-3 and the hole mutation in the heavy chain comprising a variable region that binds to PD-1.

In some aspects of the present disclosure, a bispecific antibody or antigen-binding fragment thereof for use in the methods described herein specifically binds to human TIM-3 and human PD-1 and comprises the CDRs of the AZD7789 antibody as provided in Tables 1 and 2.

TABLE 1
VH CDR Amino Acid Sequences¹

| | VH CDR1 | VH CDR2 | VH CDR3 |
|---|---|---|---|
| Anti-body | (SEQ ID NO:) | (SEQ ID NO:) | (SEQ ID NO:) |
| AZD | SYAMS | AISGSGGSTYYADSVKG | GSYGTYYGNYFEY |
| 7789 | (SEQ ID NO: 1) | (SEQ ID NO: 2) | (SEQ ID NO: 3) |
| TIM-3 | | | |
| AZD | DYGMH | YISSGSYTIYSADSVKG | RAPNSFYEYYFDY |
| 7789 | (SEQ ID NO: 4) | (SEQ ID NO: 5) | (SEQ ID NO: 6) |
| PD-1 | | | |

¹The VH CDRs in Table 1 are determined according to Kabat.

TABLE 2
VL CDR Amino Acid Sequences²

| Anti- | VL CDR1 | VL CDR2 | VL CDR3 |
|---|---|---|---|
| body | (SEQ ID NO:) | (SEQ ID NO:) | (SEQ ID NO:) |
| AZD | GGDNIGGKSVH | YDSDRPS | QVLDRRSDHFL |
| 7789 | (SEQ ID NO: 7) | (SEQ ID NO: 8) | (SEQ ID NO: 9) |
| TIM-3 | | | |
| AZD | SASSKHTNLYWSRHMY | LTSNRAT | QQWSSNP |
| 7789 | WY | (SEQ ID NO: 11) | (SEQ ID NO: 12) |
| PD-1 | (SEQ ID NO: 10) | | |

²The VL CDRs in Table 2 are determined according to Kabat.

In some aspects of the present disclosure, a bispecific antibody or antigen-binding fragment thereof for use in the methods described herein, the TIM-3 binding protein comprises Complementarity-Determining Regions (CDRs): HCDR1, HCDR2, HCDR3, LCDR1, LCDR2, and LCDR3 comprising the amino acid sequences of SEQ ID NOs: 1, 2, 3, 7, 8, and 9, respectively. In some aspects of the present disclosure, a bispecific antibody or antigen-binding fragment thereof for use in the methods described herein the TIM-3 binding protein comprises Complementarity-Determining Regions (CDRs): HCDR1, HCDR2, HCDR3, LCDR1, LCDR2, and LCDR3 comprising the amino acid sequences of SEQ ID NOs: 1, 2, 3, 7, 8, and 13, respectively.

In some aspects of the present disclosure, the TIM-3 binding domain of the bispecific antibody or antigen-binding fragment thereof for use in the methods described herein, specifically binds to a unique epitope on the IgV domain of TIM-3. The epitope on the IgV domain of TIM-3 comprises N12, L47, R52, D53, V54, N55, Y56, W57, W62, L63, N64, G65, D66, F67, R68, K69, D71, T75, and E77 of TIM-3 (SEQ ID NO: 29).

In some aspects of the present disclosure, a bispecific antibody or antigen-binding fragment thereof for use in the methods described herein, the TIM-3 binding protein further comprises a Programmed cell death protein 1 (PD-1) binding domain. In some aspects, the TIM-3 binding domain comprises a first set of Complementarity-Determining Regions (CDRs): HCDR1, HCDR2, HCDR3, LCDR1, LCDR2, and LCDR3 comprising the amino acid sequences of SEQ ID NOs: 1, 2, 3, 7, 8, and 9, respectively; and the PD-1 binding domain comprises a second set of CDRs: HCDR1, HCDR2, HCDR3, LCDR1, LCDR2, and LCDR3 comprising the amino acid sequences of SEQ ID NOs: 4, 5, 6, 10, 11, and 12, respectively.

In some aspects of the present disclosure, a bispecific antibody or antigen-binding fragment thereof for use in the methods described herein, the TIM-3 binding protein further comprises a Programmed cell death protein 1 (PD-1) binding domain. In some aspects, the TIM-3 binding domain comprises a first set of Complementarity-Determining Regions (CDRs): HCDR1, HCDR2, HCDR3, LCDR1, LCDR2, and LCDR3 comprising the amino acid sequences of SEQ ID NOs: 1, 2, 3, 7, 8, and 13, respectively; and the PD-1 binding domain comprises a second set of CDRs: HCDR1, HCDR2, HCDR3, LCDR1, LCDR2, and LCDR3 comprising the amino acid sequences of SEQ ID NOs: 4, 5, 6, 10, 11, and 12, respectively.

In some aspects of the present disclosure, a bispecific antibody or antigen-binding fragment thereof for use in the methods described herein specifically binds to human TIM-3 and PD-1 and and comprises the heavy chain variable domain (VH) and light chain variable domain (VL) of the AZD7789 antibody listed in Table 3.

TABLE 3
VH and VL amino acid sequences

| Antibody | Amino Acid Sequence (SEQ ID NO) |
|---|---|
| AZD | EVQLLESGGGLVQPGGSLRLSCAASGFTFSSYAMSWVRQAPGKGLE |
| 7789 | WVSAISGSGGSTYYADSVKGRFTISRDNSKNTLYLQMNSLRAEDTAV |
| TIM-3 | YYCARGSYGTYYGNYFEYWGQGTLVTVSS (SEQ ID NO: 14) |
| VH | |
| AZD | SYVLTQPPSVSVAPGKTARITCGGDNIGGKSVHWYQQKPGQAPVLVI |
| 7789 | YYDSDRPSGIPERFSGSNSGNTATLTISRVEAGDEADYYCQVLDRRSD |
| TIM-3 | HFLFGGGTKLTVL (SEQ ID NO: 17) |
| VL | |
| AZD | EVQLVESGGGLVQPGGSLRLSCAASGFTFSDYGMHWVRQAPGKGLE |
| 7789 | WVAYISSGSYTIYSADSVKGRFTISRDNAKNSLYLQMNSLRAEDTAV |
| PD-1 | YYCARRAPNSFYEYYFDYWGQGTTVTVSS (SEQ ID NO: 19) |
| VH | |
| AZD | QIVLTQSPATLSLSPGERATLSCSASSKHTNLYWSRHMYWYQQKPGQ |
| 7789 | APRLLIYLTSNRATGIPARFSGSGSGTDFTLTISSLEPEDFAVYYCQQW |
| PD-1 | SSNPFTFGQGTKLEIK (SEQ ID NO: 21) |
| VL | |

In some aspects of the present disclosure, a bispecific antibody or antigen-binding fragment thereof for use in the methods described herein, the TIM-3 binding protein comprises a first heavy chain variable domain (VH) comprising the amino acid sequence of SEQ ID NO: 14, a first light chain variable domain (VL) comprising the amino acid sequence of SEQ ID NO: 17, a second heavy chain VH comprising the amino acid sequence of SEQ ID NO: 19, and a second light chain VL comprising the amino acid sequence of SEQ ID NO: 21.

In some aspects of the present disclosure, a bispecific antibody or antigen-binding fragment thereof for use in the methods described herein specifically binds to human TIM-3 and PD-1 and comprises the Heavy Chain (HC) and Light Chain (LC) of the AZD7789 antibody listed in Table 4.

TABLE 4
Full-length heavy chain amino acid sequences

| Antibody | Amino Acid Sequence (SEQ ID NO) |
|---|---|
| AZD | EVQLLESGGGLVQPGGSLRLSCAASGFTFSSYAMSWVRQAPGKGLE |
| 7789 | WVSAISGSGGSTYYADSVKGRFTISRDNSKNTLYLQMNSLRAEDTAV |
| TIM-3 | YYCARGSYGTYYGNYFEYWGQGTLVTVSSASTKGPSVCPLAPSSKST |
| HC | SGGTAALGCLVKDYFPEPVTVSWNSGALTSGVHTFPAVLQSSGLYSL |
| | SSVVTVPSSSLGTQTYICNVNHKPSNTKVDKRVEPKSVDKTHTCPPCP |
| | APEFEGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPASIEKTISKAKGQPREPQVYTLPPCREEMTKNQVSLWCLVK |
| | GFYPSDIAVEWESNGQPENNYKTTPPVLDSDGSFFLYSKLTVDKSRW |
| | QQGNVFSCSVMHEALHNHYTQKSLSLSPGK (SEQ ID NO: 15) |
| AZD | SYVLTQPPSVSVAPGKTARITCGGDNIGGKSVHWYQQKPGQAPVLVI |
| 7789 | YYDSDRPSGIPERFSGSNSGNTATLTISRVEAGDEADYYCQVLDRRSD |
| TIM-3 | HFLFGGGTKLTVLGQPKAAPSVTLFPPCSEELQANKATLVCLISDFYP |
| LC | GAVTVAWKADSSPVKAGVETTTPSKQSNNKYAASSYLSLTPEQWKS |
| | HRSYSCQVTHEGSTVEKTVAPTEVS (SEQ ID NO: 18) |
| AZD | EVQLVESGGGLVQPGGSLRLSCAASGFTFSDYGMHWVRQAPGKGLE |
| 7789 | WVAYISSGSYTIYSADSVKGRFTISRDNAKNSLYLQMNSLRAEDTAV |
| PD-1 | YYCARRAPNSFYEYYFDYWGQGTTVTVSSASTKGPSVFPLAPSSKST |
| HC | SGGTAALGCLVKDYFPEPVTVSWNSGALTSGVHTFPAVLQSSGLYSL |
| | SSVVTVPSSSLGTQTYICNVNHKPSNTKVDKRVEPKSCDKTHTCPPCP |
| | APEFEGGPSVFLFPPKPKDTLMISRTPEVTCVVVDVSHEDPEVKFNWY |
| | VDGVEVHNAKTKPREEQYNSTYRVVSVLTVLHQDWLNGKEYKCKV |
| | SNKALPASIEKTISKAKGQPREPQVCTLPPSREEMTKNQVSLSCAVKG |
| | FYPSDIAVEWESNGQPENNYKTTPPVLDSDGSFFLVSKLTVDKSRWQ |
| | QGNVFSCSVMHEALHNHYTQKSLSLSPGK (SEQ ID NO: 20) |
| AZD | QIVLTQSPATLSLSPGERATLSCSASSKHTNLYWSRHMYWYQQKPGQ |
| 7789 | APRLLIYLTSNRATGIPARFSGSGSGTDFTLTISSLEPEDFAVYYCQQW |
| PD-1 | SSNPFTFGQGTKLEIKRTVAAPSVFIFPPSDEQLKSGTASVVCLLNNFY |
| LC | PREAKVQWKVDNALQSGNSQESVTEQDSKDSTYSLSSTLTLSKADYE |
| | KHKVYACEVTHQGLSSPVTKSFNRGEC (SEQ ID NO: 22) |

In some aspects of the present disclosure, a bispecific antibody or antigen-binding fragment thereof for use in the methods described herein, the TIM-3 binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22.

In some aspects of the present disclosure, a bispecific antibody or antigen-binding fragment thereof for use in the methods described herein, the TIM-3 binding protein comprises a first heavy chain comprising the amino acid sequence of SEQ ID NO: 23, a first light chain comprising the amino acid sequence of SEQ ID NO: 24, a second heavy chain comprising the amino acid sequence of SEQ ID NO: 23, and a second light chain comprising the amino acid sequence of SEQ ID NO: 24.

In some aspects of the present disclosure, a bispecific antibody or antigen-binding fragment thereof for use in the methods described herein, the TIM-3 binding protein comprises a first heavy chain comprising the amino acid sequence of SEQ ID NO: 25, a first light chain comprising the amino acid sequence of SEQ ID NO: 26, a second heavy chain comprising the amino acid sequence of SEQ ID NO: 25, and a second light chain comprising the amino acid sequence of SEQ ID NO: 26.

In some aspects, the TIM-3 binding protein of the bispecific antibody or antigen-binding fragment thereof for use in the methods described herein comprises an aglycosylated Fc region. In some aspects, the TIM-3 binding protein comprises a deglycosylated Fc region. In some aspects, the TIM-3 binding protein comprises an Fc region which has reduced fucosylation or is afucosylated.
5.6 Methods of Treatment

In some aspects, the disclosure provides a method of treating non-small cell lung cancer (NSCLC) in a subject. In some aspects, the disclosure provides a method of treating NSCLC in a subject having advanced or metastatic NSCLC.

In some aspects, the method of treating NSCLC in a subject having advanced or metastatic NSCLC comprises administering to the subject a bispecific binding protein comprising a PD-1 binding domain and a TIM-3 binding domain, wherein the bispecific binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22, and wherein the subject has 10 acquired resistance. In some aspects, the TIM-3 binding domain of the present disclosure specifically binds to the C′C″ and DE loops of the IgV domain of TIM-3.

In some aspects, the disclosure provides a method of inhibiting growth of a non-small cell lung tumor in a subject having an advanced or metastatic tumor. In some aspects of the method of inhibiting growth of a non-small cell lung tumor in a subject having an advanced or metastatic tumor, the method comprises administering to the subject a bispecific binding protein comprising a PD-1 binding domain and a TIM-3 binding domain, wherein the bispecific binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22, and wherein the subject has 10 acquired resistance, wherein. In some aspects, the TIM-3 binding domain specifically binds to the C′C″ and DE loops of the IgV domain of TIM-3.

In some aspects, the TIM-3 binding domain of the bispecific binding protein comprising a PD-1 binding domain and a TIM-3 binding domain described herein specifically binds to epitopes on the IgV domain of TIM-3 and the epitopes comprise N12, L47, R52, D53, V54, N55, Y56, W57, W62, L63, N64, G65, D66, F67, R68, K69, D71, T75, and E77 of TIM-3 (SEQ ID NO: 29).

In some aspects, the NSCLC is squamous or non-squamous NSCLC.

In some aspects, the disclosure provides a method of treating a cancer in a subject with IO acquired resistance. In some aspects, the method of treating a cancer in a subject with IO acquired resistance comprises administering to the subject a TIM-3 binding protein comprising a TIM-3 binding domain, wherein the TIM-3 binding domain specifically binds to the C′C″ and DE loops of the IgV domain of TIM-3. In some aspects, the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, lung cancer, esophageal cancer, gastric cancer, biliary tract tumors, urothelial carcinoma, Hodgkin lymphoma, non-hodgkin lymphoma, myelodysplastic syndrome, and acute myeloid leukemia. In some aspects, the subject is a human. In some aspects, the the subject has documented Stage III which is not amenable to curative surgery or radiation, or Stage IV non-small cell lung carcinoma (NSCLC).

In some aspects, administration of the TIM-3 binding protein results in inhibition of tumor growth in the subject.

The following examples are offered by way of illustration and not by way of limitation.

### 6. EXAMPLES

The examples in this Section(i.e., Section 6) are offered by way of illustration, and not by way of limitation.

### 6.1 Example 1

### TIM-3 IgV Domain Characterization

The TIM-3 IgV domain interactions with antigen binding fragments of the anti-TIM3 #62 monoclonal antibody (“#62” or “clone 62”) were investigated. Clone 62 is the parent of anti-TIM-3 antibody O13-1, which is an affinity mature variant of clone 62. The sequences of mAb O13-1 and clone 62 are disclosed in U.S. Pat. No. 10,457,732, which is incorporated by reference herein, in its entirety.

### Crystallization, Data Collection, and Structure Determination

To obtain the co-crystal structure of the TIM-3 IgV domain with antigen-binding fragments (Fabs), all proteins were expressed in mammalian cells and purified to homogeneity. Purified TIM-3 IgV domain and Fabs (one at a time) were incubated at a slight excess of IgV domain, followed by size exclusion purification of the complex. Crystallization of the complexes was performed at room temperature. The X-ray diffraction data was collected at the Stanford Synchrotron Radiation Lightsource (SSRL, Menlo Park, Calif., USA). Structures of the complexes were solved by molecular replacement.

The crystal structure of the Fab of anti-TIM3 antibody #62, as bound to the IgV domain of TIM-3, was determined at a resolution of 2.2 A. Both heavy and light chains of the Fab interacted with the antigen. Two chains of the Fab create an interface area of 815A², with 365A² contributed by the light chain and 450A² contributed by the heavy chain. In total, there are 27 amino acids from both chains of Fab participating in the interaction and 19 amino acids from the IgV domain of TIM-3. Some of the TIM-3 amino acids interacted with both chains of the Fab.

The following amino acids of the IgV domain belong to the interface and/or participate in the interactions with the heavy chain of anti-TIM3 antibody #62: Fab: N12, L47, D53, V54, N55, Y56, W57, W62, L63, N64, G65, D66, F67, T75, and E77. Among these, amino acids N12, L63 (main chain), and E77 establish hydrogen bonds with CDRs 2 and 3 of the heavy chain.

Hydrogen bonds created between the
heavy chain of anti-TIM3 antibody #62
and the IgV domain of TIM-3

| | | | | | |
|---|---|---|---|---|---|
| 1 | G:ASN | 12 [ND2] | 3.71 | H:TYR | 104 [OH] |
| 2 | G:LEU | 63 [N] | 3.15 | H:GLY | 102 [O] |
| 3 | G:LEU | 63 [O] | 2.97 | R:TYR | 104 [N] |
| 4 | G:GLU | 77 [OE2] | 2.57 | H:SER | 54 [OG] |

In the Fab of the heavy chain of anti-TIM3 antibody #62, the following amino acids belong to the interface and/or participate in the interaction with the IgV domain of TIM-3: S30, S31, Y32, and A33 (all belong to CDR1 of the heavy chain), S52, G53, S54, G56, S57 (all belong to CDR2 of the heavy chain), S100, Y101, G102, T103, Y104, Y105, N107, and Y108 (all belong to CDR3 of the heavy chain).

The following amino acids of the IgV domain belong to the interface and/or participate in the interaction with the Fabs in the light chain of anti-TIM3 antibody: R52, D53, L63, N64, G65, D66, F67, R68, K69, D71.

| Hydrogen bonds created between the | | | | | |
|---|---|---|---|---|---|
| light chain of anti-TIM3 antibody #62 | | | | | |
| and the IgV domain of TIM-3 | | | | | |
| 1 | G:ARG | 68 [NH1] | 3.57 | L:TYR | 49 [O] |
| 2 | G:LYS | 69 [NZ] | 2.72 | L:GLY | 28 [O] |
| 3 | G:LYS | 69 [NZ] | 2.99 | L:ASP | 50 [OD2] |
| 4 | G:ASP | 53 [OD2] | 3.22 | L:TYR | 48 [OH] |
| 5 | G:ASN | 64 [O] | 2.82 | L:ARG | 92 [NE] |
| 6 | G:ASP | 66 [OD1] | 2.58 | L:SER | 31 [OG] |
| 7 | G:ASP | 66 [OD2] | 2.91 | L:SER | 31 [N] |
| Salt bridges created between the | | | | | |
| heavy chain of anti-TIM3 antibody #62 | | | | | |
| and the IgV domain of TIM-3 | | | | | |
| 1 | G:ARG | 52 [NE] | 3.97 | L:ASP | 52 [OD2] |
| 2 | G:ARG | 68 [NH2] | 3.02 | L:ASP | 52 [OD1] |
| 3 | G:ARG | 68 [NH2] | 2.83 | L:ASP | 52 [OD2] |
| 4 | G:LYS | 69 [NZ] | 2.99 | L:ASP | 50 [OD2] |

In the Fab of the light chain of anti-TIM3 antibody #62, the following amino acids belong to the interface and/or participate in the interaction with the IgV domain of TIM-3: G28, G29, K30, and S31 (all belong to CDR1 of the light chain), Y48, Y49, D50, S51, D52, R53 (all belong to CDR2 of the light chain), and R92 (belongs to CDR3 of the light chain).

This demonstrates that the antigen-binding fragments of anti-TIM3 antibody #62 bind the IgV domain from the side opposite of phosphatidylserine binding. This binding does not introduce changes into the fold or structure of the IgV domain of TIM-3. Models of the IgV domain from this structure align one with bound phosphatidylserine with a root mean square deviation of 0.7A. The interaction interface of anti-TIM3 antibody #62 with the IgV domain of TIM-3 does not include a glycosylated asparagine at position of 78 nor does it attach to the carbohydrate itself.

### 6.2 Example 2

### Binding of TIM-3 With Phosphatidylserine

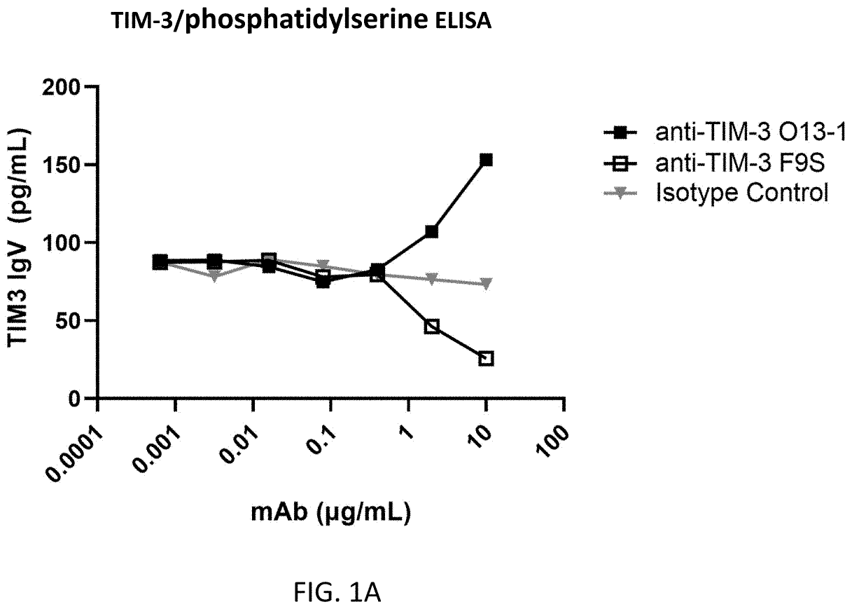

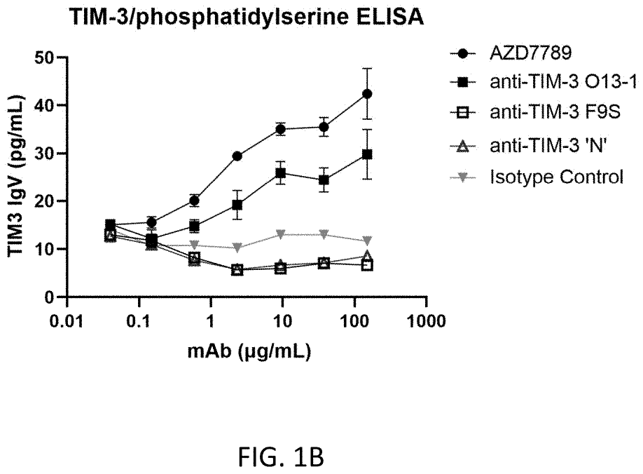

Phosphatidylserine was plated at 30 μg/mL onto a multi-array 96 well plate (Meso Scale Discovery) and left to evaporate overnight. Plates were blocked with 1% bovine serum albumin. Drugs were titrated by a 7 point curve with a 5-fold serial dilution starting at 10 μg/mL. Then, 5 μg/mL of TIM-3 IgV conjugated to SULFO-tag (Meso Scale Discovery) was preincubated with drug for 15 minutes before addition to the plate. After a 1.5 hour incubation period, the plates were washed and an electrochemiluminescence signal was detected on a MESO SECTOR 5600 instrument (Meso Scale Discovery) (FIG. 1A).

The data provided in FIG. 1A demonstrate that the parental anti-TIM-3 mAb to AZD7789 (i.e., mAb O13-1) increases binding of TIM-3 with phosphatidylserine, as compared to an isotype control. Conversely, titration of the anti-TIM-3 mAb F9S blocks the interaction of TIM-3 with phosphatidylserine. Overall, this data indicates that antibodies that bind to differing epitopes of TIM-3 can differentially modulate the interaction of TIM-3 and phosphatidylserine.

Next, phosphatidylserine was plated at 30 μg/mL onto a multi-array 96 well plate (Meso Scale Discovery) and left to evaporate overnight. Plates were blocked with 1% bovine serum albumin. Drugs were titrated by a 7 point curve with a 4-fold serial dilution starting at 150 μg/mL. Then, 1.67 μg/mL of TIM-3 IgV conjugated to SULFO-tag (Meso Scale Discovery) was added per well immediately post drug addition. After a two hour incubation period, plates were washed and an electrochemiluminescence signal was detected on a MESO SECTOR 5600 instrument (Meso Scale Discovery). Duplicate wells were evaluated per treatment. (FIG. 1B).

This data demonstrates that monovalent engagement at the C′CC″/DE epitope of TIM-3, as confirmed by AZD7789 versus anti-TIM-3 013-1 binding, is sufficient to increase TIM-3 interaction with phosphatidylserine as compared to an isotype control. Conversely, two independently derived anti-TIM-3 antibodies that bind to the CC′/FG of TIM-3 (mAb F9S and mAb ‘N’) blocked the interaction of TIM-3 with phosphatidylserine. Overall, this data indicates that antibodies that bind to differing epitopes of TIM-3 can differentially modulate the interaction of TIM-3 and phosphatidylserine and this effect can be observed through monovalent and bivalent engagement.

### 6.3 Example 3

### TIM3 IgV Binding to Killed A375 Melanoma Cell Line

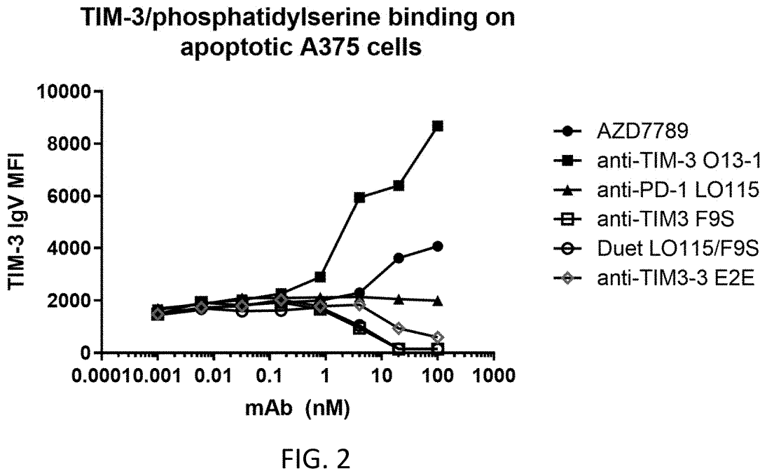

A375 melanoma cells were killed with 1 μM/mL staurosporine for 24 hours. The next day cells were washed and two hundred thousand cells were plated per well. Drug was titrated by a 5-fold serial dilution and co-incubated with 10 μg/mL TIM-3 IgV for 45 minutes. The drug/TIM3 IgV mixture was then incubated with apoptotic A375 cells. After 45 minutes, cells were washed with cold buffer and fixed with 4% PFA for 20 minutes. Data was acquired on a BD Symphony A2 and analyzed via flowjo. Graphs were generated using PRISM. Duplicate wells were evaluated per treatment. (FIG. 2).

The data presented in FIG. 2 show that AZD7789 and clone O13-1 enhance binding of soluble TIM-3 IgV to apoptotic melanoma cells, while anti-PD-1 LO115 does not. Anti-TIM-3 E2E and duet LO115/F9S antibodies decrease engagement of TIM-3 with apoptotic cells.

### 6.4 Example 4

### Jurkat Cell Lines Engineered to Express Human TIM-3

A Jurkat cell line was engineered to express human TIM-3 (h-TIM-3 Jurkat cells).

Two hundred thousand cells were plated per well. Drug was titrated by a 9 point 4-fold serial dilution starting at 10 μg/mL. Immediately following drug addition, cells were stimulated with soluble anti-CD3 (2.5 μg/mL) and anti-CD28 (0.5 μg/mL). After 24 hours, the supernatant was collected and IL-2 was evaluated by electrochemiluminescence detection utilizing Meso Scale Discovery's human IL-2 Tissue Culture kit. Duplicate wells were evaluated per treatment.

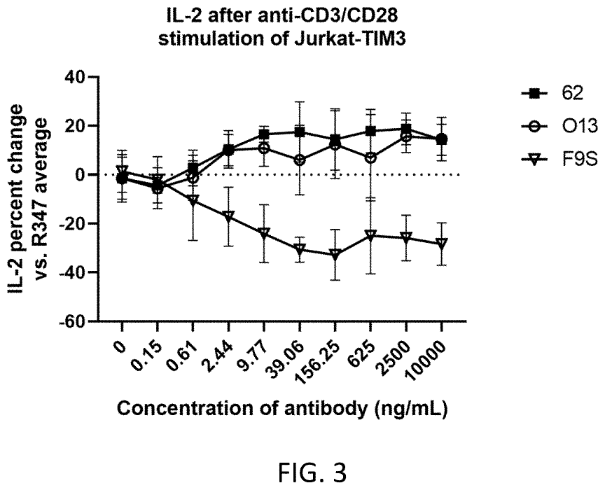

As shown in FIG. 3, the non-lead optimized and lead optimized parental anti-TIM-3 mAb to AZD7789 (anti-TIM-3 antibody #62, and O13 respectively) increases IL-2 production of the h-TIM-3 Jurkat cells, as compared to an isotype control upon T cell stimulation (Error bars represent SEM). Conversely, anti-TIM-3 mAb 41 or F9S, reduces IL-2 production under the same stimulation conditions. Overall, this data indicates that antibodies that bind to differing epitopes of TIM-3 can elicit differential outcomes in the human-TIM3 Jurkat stimulation assay. The one amino acid change between the non-lead optimized clone 62 and lead optimized clone 13 does not change the functional outcome in this Jurkat stimulation assay.

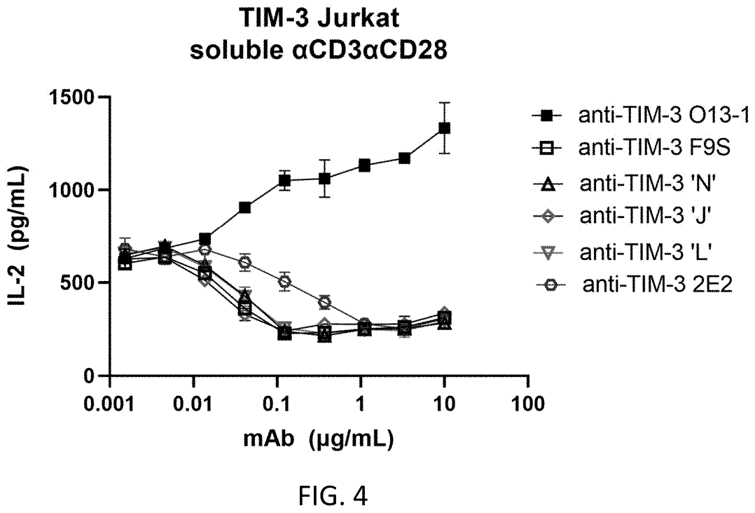

In a separate study, two hundred thousand h-TIM-3 Jurkat cells were plated per well. Drug was titrated by a 9 point 3-fold serial dilution starting at 10 mg/mL. Immediately following drug addition, cells were stimulated with anti-CD3 (1 μg/mL)/anti-CD28 (0.5 μg/mL). After 24 hours, the supernatant was collected and IL-2 was evaluated by electrochemiluminescence detection utilizing Meso Scale Discovery's human IL-2 Tissue Culture kit. (FIG. 4). Duplicate wells were evaluated per treatment. Error bars represent SEM. The comparator anti-TIM-3 antibodies ‘N’; ‘J’; and ‘L’ were derived from patent sequences. The anti-TIM-3 antibody 2E2 is commercially available (Leaf purified anti-human CD366, Biolegend).

As shown in FIG. 4, titration of the parental anti-TIM-3 mAb O13-1 increases IL-2 production of the h-TIM-3 Jurkat cells upon T cell stimulation. Conversely, titration of all of the other anti-TIM-3 mAbs evaluated in this assay reduce IL-2 production under the same stimulation conditions. Overall, this data indicates that antibodies that bind to differing epitopes of TIM-3 can elicit differential outcomes in the human-TIM-3 Jurkat stimulation assay.

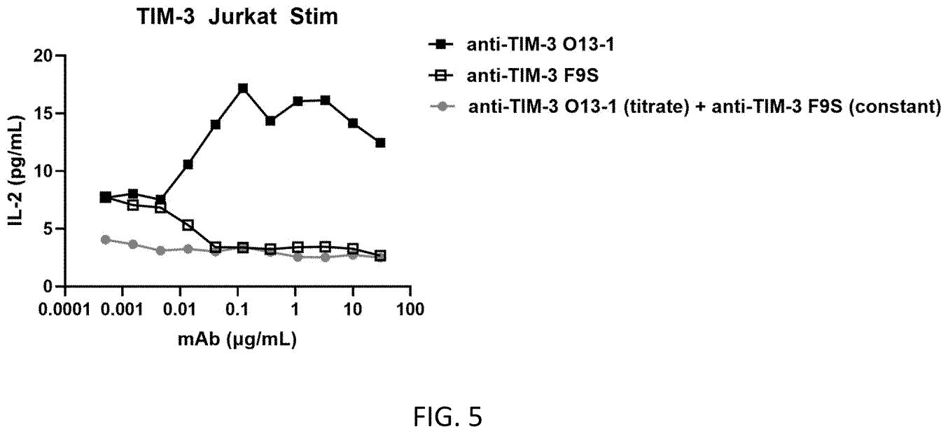

In another study with the h-TIM-3 Jurkat cells, drug was titrated by an 11 point 3-fold serial dilution starting at 30 μg/mL. For the “anti-TIM-3 O13-1 (titrate)+anti-TIM-3 F9S (constant)” treatment group, cells were incubated with a constant concentration of anti-TIM-3 mAb F9S (10 μg/mL) prior to the titration of anti-TIM-3 mAb O13-1. Following drug addition, cells were stimulated with anti-CD3 (1 μg/mL)/anti-CD28 (0.5 μg/mL). After 24 hours, supernatant was collected, and IL-2 was evaluated by electrochemiluminescence detection utilizing Meso Scale Discovery's human IL-2 Tissue Culture kit (FIG. 5).

As shown in FIG. 5, the observed increase of IL-2 from the stimulated h-TIM-3 Jurkat cells following the addition of anti-TIM-3 mAb O13-1 is ablated when cells are cultured in a high concentration of anti-TIM-3 mAb F9S, which blocks TIM-3 interaction with phosphatidylserine. This data suggests that anti-TIM-3 mAb O13-1 induced IL-2 production is dependent on interaction of TIM-3 with phosphatidylserine, and abrogation of this interaction prevents the enhanced IL-2 secretion.

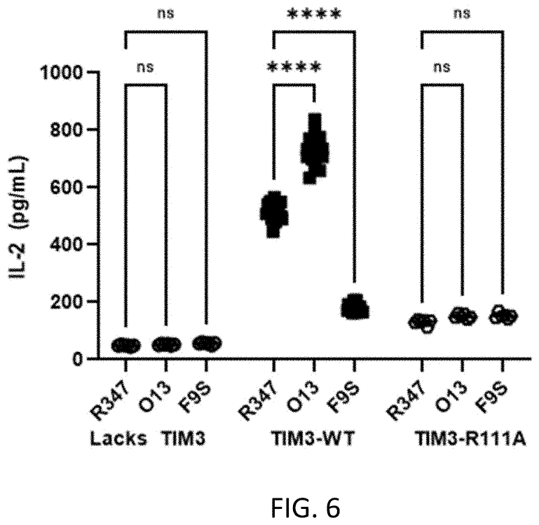

Next, parental Jurkat T cells were compared to two Jurkat cell lines genetically engineered to express wildtype and mutant (R111A) versions of human TIM-3 R111 is a critical residue for TIM-3 binding to phosphatidylserine. R111A mutation in TIM-3 abrogates phosphatidylserine binding to TIM-3 (Gandhi et al., Scientific Reports 2018; 8:17512; Nakayama et al., Blood, 2009). Following drug addition, cells were stimulated with anti-CD3 (2.5 μg/mL)/anti-CD28 (0.5 μg/mL). After 24 hours, the supernatant was collected and IL-2 was evaluated by electrochemiluminescence detection utilizing Meso Scale Discovery's human IL-2 Tissue Culture kit (FIG. 6). Data was compiled from three independent experiments treated at 50 nM. Error bars represent SEM. ****, p<0.0001.

The data presented in FIG. 6 indicate that TIM-3 expression, along with engagement to phosphatidylserine is required for anti-TIM-3 mAb O13-1 mediated increase of IL-2 production from TIM-3 expressing Jurkat cells following stimulation.

### 6.5 Example 5

### IFN-γ Secretion in Primary Human T Cells

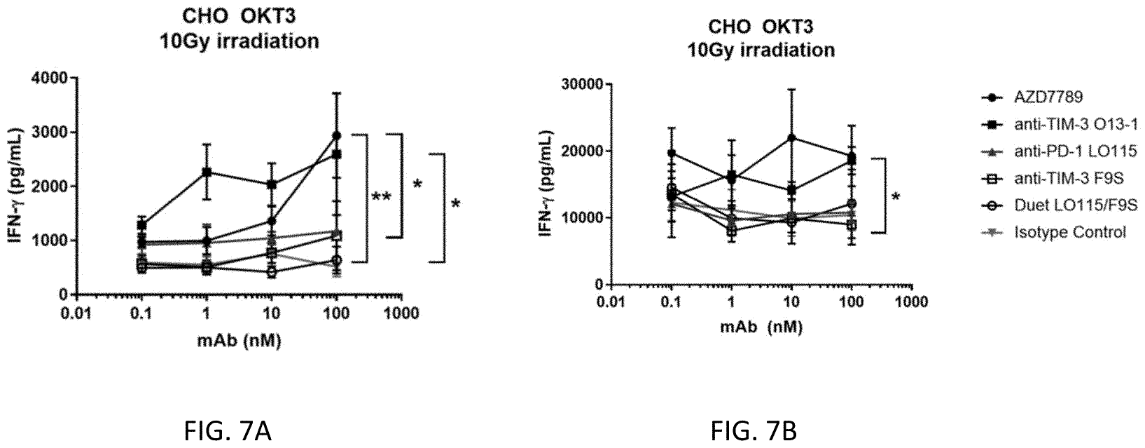

Fresh peripheral blood mononuclear cells (PBMC) from two healthy donors were plated at 40,000 cells/well. Drug was titrated by a 4 point 10-fold serial dilution starting at 100 nM. A Chinese hamster ovary (CHO) cell line was engineered to express human anti-CD3 OKT3 single-chain variable fragment (scFv) on the cell surface. The CHO-OKT3 cells were irradiated (10 Gy) to induce apoptosis and plated at 5,000 per well. Cells were co-cultured for three days. The supernatant was then collected and IFN-γ was evaluated by electrochemiluminescence detection utilizing Meso Scale Discovery's human IFN-γ Tissue Culture kit (FIGS. 7A and 7B). Error bars represent SEM of triplicate wells. **, p<0.01; *, p<0.05.

The data shown in FIGS. 7A and 7B indicate that AZD7789 and its parental bivalent anti-TIM-3 mAb, O13-1, enhance IFN-γ secretion of primary human T cells stimulated in the context of cellular apoptosis. The same is not true for phosphatidylserine blocking anti-TIM-3 molecules in an antibody or bispecific format.

### 6.6 Example 6

### Effect of AZD7789 on Dendritic Cell Efferocytosis of Apoptotic Tumor Cells

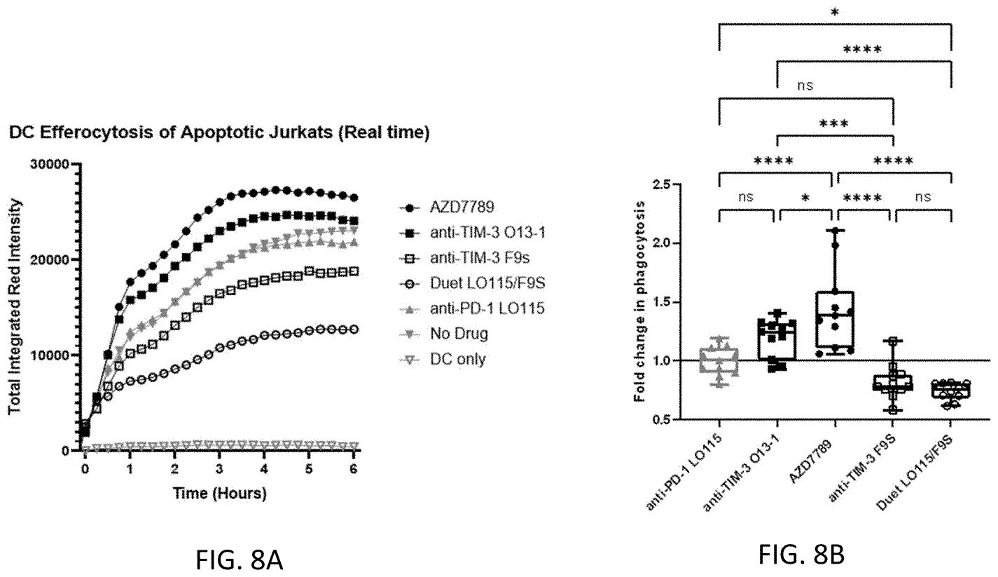

Human dendritic cells (DC) were generated from freshly isolated monocytes cultured in the presence of 100 ng/mL IL-4 and 100 ng/mL Granulocyte-Macrophage Colony Stimulating Factor (GM-CSF) for 6 days. To induce apoptosis, Jurkat cell lines were treated with 100 mM staurosporine for 24 hours. Apoptotic Jurkat cells were then labeled with 1 ng/mL Incucyte®pHrodo®Red dye. Apoptotic cells were co-cultured at a 4:1 ratio with monocyte derived DC in the presence of the test drugs. Plates were placed inside the Incucyte® S3 Live-Cell Imaging system. Images were taken every 15 minutes over a 24-hour period. Red fluorescence was measured and analyzed using Incucyte® S3 2018B software. Graphical depictions of data were performed using GraphPad Prism version 8.04.02 for Windows (GraphPad Software). FIG. 8A shows an example of representative data from one experiment generated using Incucyte® S2 2018B software. FIG. 8B shows a graphical representation of data compiled from 10+independent experiments. The fold change in efferocytosis in FIG. 8B was determined from the no drug treatment group. ****, p<0.0001; ***, p<0.001; *, p<0.05.

The data shown in FIGS. 8A and 8B demonstrate that AZD7789 can enhance dendritic cell efferocytosis of apoptotic tumor cells. In contrast, an antibody targeting the phosphatidylserine binding cleft of TIM-3 (mAb F9S) showed a reduced effect compared to control groups.

### 6.7 Example 7

### The effect of AZD7789 on DC Cross Presentation of Tumoral Antigen

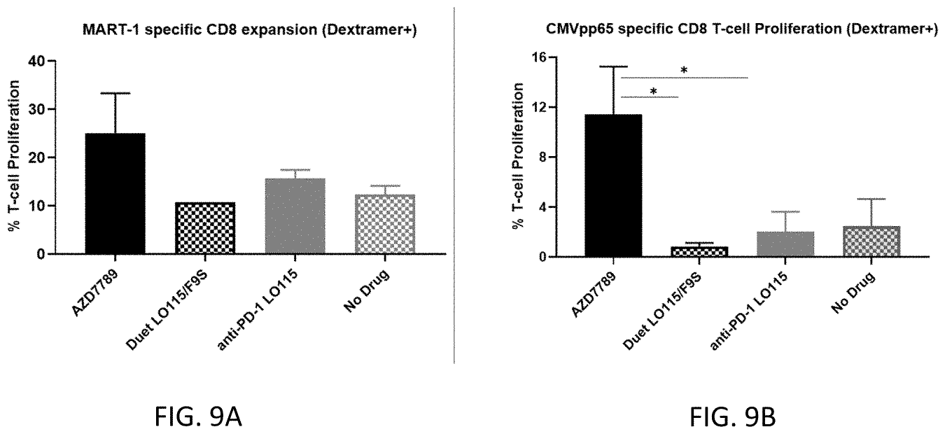

Two Jurkat cell lines were engineered to express either human MART-1 or CMVpp65 antigens, respectively. These cell lines served as the tumor cells within the assay. To induce apoptosis, the MART-1 and CMVpp65 Jurkat cell lines were treated with 100 mM staurosporine for 24 hours. Human dendritic cells (DC) were generated from freshly isolated monocytes cultured in the presence of 100 ng/mL IL-4 and 100 ng/mL GM-CSF for six days. Monocytes were isolated from HLA-A*02 positive healthy donor blood. Dendritic cells were co-cultured (1:4 ratio) with apoptotic MART-1 or CMVpp65 Jurkat cells in the presence of test articles and incubated for 24 hours to allow efferocytosis and antigen processing. Donor matched antigen-specific T cells were generated from frozen PBMC and peptide stimulated for seven days using either antigen peptide MART-1 (Leu26)—HLA-A*0201 (ELAGIGILTV) or antigen peptide CMV pp65—HLA-A*0201 (NLVPMVATV). Following the 24 hour DC efferocytosis of MART-1 or CMVpp65 Jurkat cells, the remaining apoptotic Jurkat cells were removed by washing wells 2 times with media. Antigen-specific T cells were labeled with CellTrace proliferation dye and co-cultured with DC at a 1:4 ratio (DC:T cell) for seven days. After seven days, T cells were stained for CD3, CD8, and antigen specificity using dextramer: HLA-A*0201/NLVPMVATV—antigen: pp65 or dextramer: HLA-A*0201/ELAGIGLTV—antigen MART-1. Proliferation of antigen-specific T cells was determined by flow cytometry and analyzed using FlowJo software. (FIGS. 9A and 9B). Bar graphs depict duplicate wells for the MART-1 Jurkat cell experiment, and triplicate wells for the CMVpp65 Jurkat cell experiment, error bars represent SEM; *, p<0.05.

The data presented in FIGS. 9A and 9B indicate that AZD7789 can enhance DC cross-presentation of tumor antigen to T cells. This effect is different from a similar modality blocking the phosphatidylserine binding site on TIM-3 (Duet LO115/F9S). This example demonstrates that AZD7789 can improve anti-tumor responses via enhanced DC cross-presentation to antigen-specific T cells.

### 6.8 Example 8

### Comparison of Tumor Growth Inhibition and Survival for Anti-PD-1 Versus AZD7789

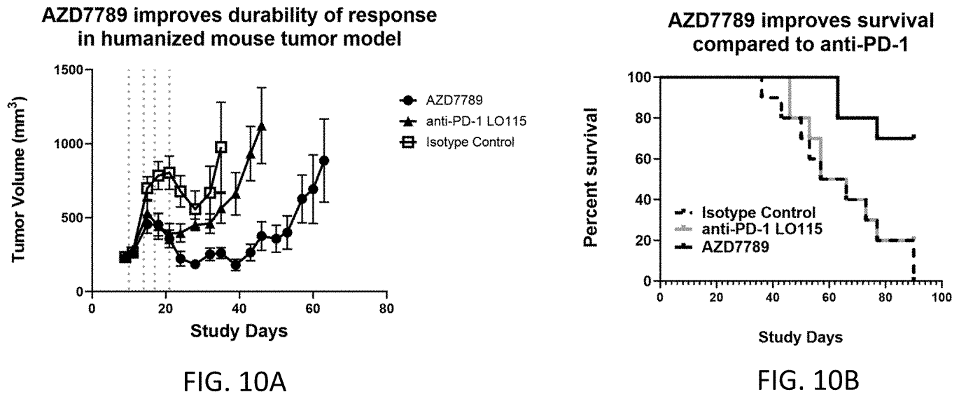

Immunodeficient NOD.Cg-Prkdcscid IL2rgtm1Wjl/SzJ (NSG) mice were subcutaneously engrafted on study day 0 with 2×10⁶ OE21-10xGSV3 cells, a human oesophageal squamous cell carcinoma engineered to express viral peptides of interest. Seven days later, viral peptide reactive CD8+ T cells originating from healthy donor PBMC were intravenously administered (1×10⁶/mouse). The anti-PD-1 mAb LO115 or anti-PD-1/anti-TIM3 mAb AZD7789 was intraperitoneally administered starting on study day 10, and mice received 4 total doses (10 mg/kg), with a 2 to 3 day interval between dosing. Tumor volume was continuously monitored. Mice were sacrificed when tumor size reached 2000 mm³. The tumor volume graph (FIG. 10A) shows treatment comparisons between isotype control, AZD7789, and anti-PD-1 mAb LO115; n=8 mice/treatment and all treatments were dosed at 10 mg/kg. Survival of mice among treatment groups is showed in FIG. 10B.

These results in FIG. 10A-B demonstrate that in an antigen-specific humanized mouse tumor model, treatment of AZD7789 delays tumor growth and enhances survival compared to mice continuously treated with anti-PD-1 or isotype control. This suggests that treatment with AZD7789 may benefit patients to a greater extent than anti-PD-1 therapy.

### 6.9 Example 9

### Effect of Administration of AZD7789 on Tumor Growth

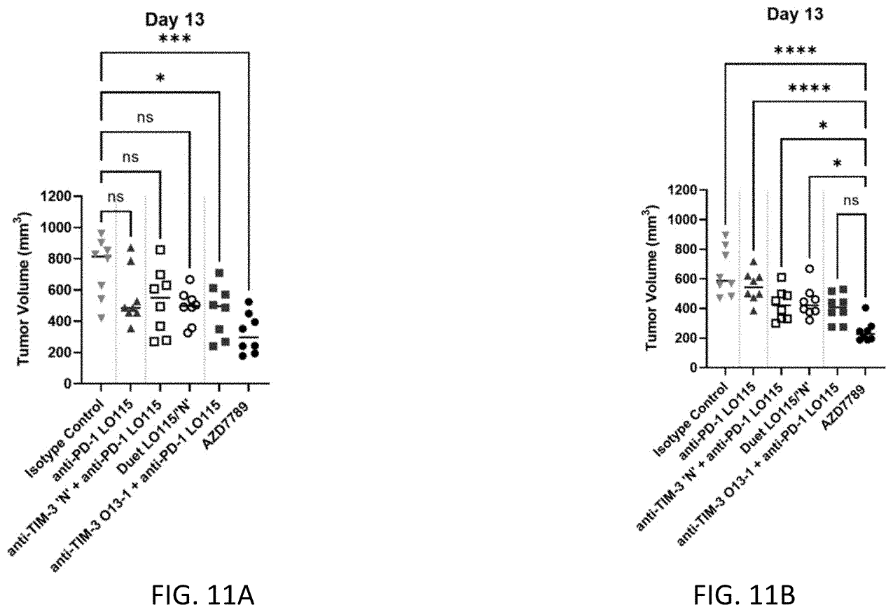

Forty-eight immunodeficient NOD.Cg-Prkdc^{scid} IL2rg^{tm1Wjl}/SzJ (NSG) mice were subcutaneously engrafted on Day 1 with 2×10⁶ 0E21-10xGSV3 cells, a human oesophageal squamous cell carcinoma engineered to express viral peptides of interest. Tumor antigen specific CD8+ T cells originating from PBMC of two healthy donors (D203517 and D896) were intravenously administered (1×10⁶/mouse) on Day 7. Mice were randomized by tumor volume on Day 8 into 6 different treatment groups, with 8 mice per group. Test and control articles were intraperitoneally administered starting on Day 9, and mice received 4 total doses (each at 10 mg/kg). FIGS. 11A and 11B depict the tumor volume on Day 13 for two independent studies with different T cell donors (D203517 and D896). A comparison between the tumor volume of the isotype control and all other drug treatments was made, and intergroup differences were analyzed for statistical significance by a one-way ANOVA, Tukeys multiple comparison test. Each symbol represents the fold-change in tumor volume from baseline to the day of the third dose (Day 13) of test or control articles. The horizontal bars represents the intragroup arithmetic mean tumor volume. ****, p<0.0001; ***, p<0.001; *, p<0.05.

The data shown in FIGS. 11A and 11B demonstrates that treatment with AZD7789 results in decreased tumor growth, as compared to treatment with anti-PD-1 antibodies alone, or treatment with a combination of anti-PD-1 antibody and a phosphatidylserine blocking anti-TIM-3 molecule. This trend was observed across two donors.

### 6.10 Example 10

### Effect of AZD7789 on IFN-γ Secretion of Ex-Vivo Stimulated Tumor Infiltrating Lymphocytes Previously Exposed to Anti-PD-1 Therapy in a Humanized Mouse Tumor Model

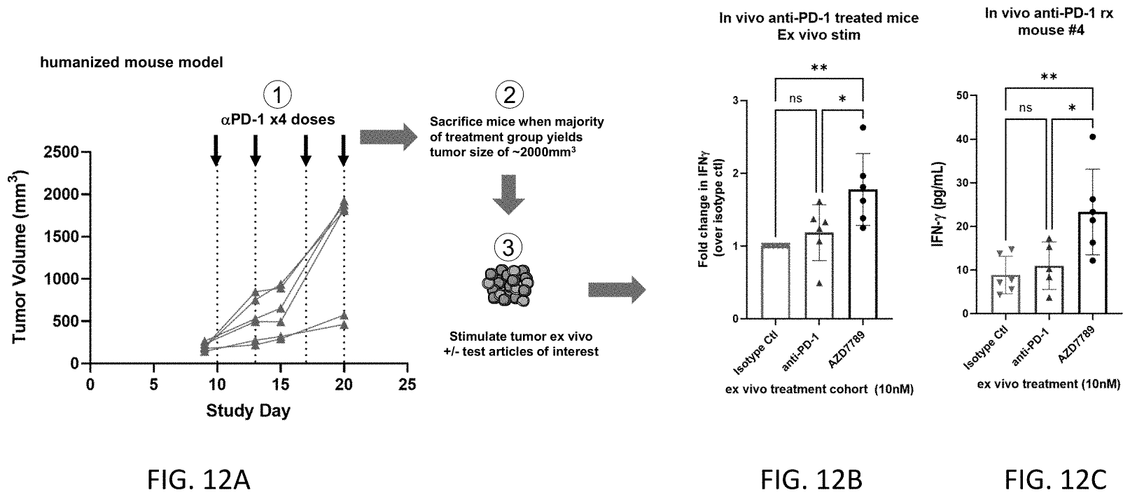

Immunodeficient NOD.Cg-Prkdcscid IL2rgtm1Wjl/SzJ (NSG) mice were subcutaneously engrafted on study day 0 with 2×10⁶ OE21-10xGSV3 cells, a human oesophageal squamous cell carcinoma engineered to express viral peptides of interest. Seven days later, viral peptide reactive CD8+ T cells originating from healthy donor PBMC were intravenously administered (1×10⁶/mouse). The anti-PD-1 mAb LO115 was intraperitoneally administered starting on study day 10, and mice received 4 total doses (10 mg/kg), with a 2 to 3 day interval between dosing. Tumor volume was continuously monitored. Mice were sacrificed when tumor size reached 2000 mm³. Tumors were disassociated into single cell suspension. Cells were centrifuged with a ficoll gradient to retain viable cells and plated at 0.1×10⁶ per well. Test and control articles (10 nM), recombinant human IL-2 (20 IU/mL), and 0.02×10⁶ T2 cells pulsed with 1.5 mg/mL GILGFVFTL peptide were added to the respective wells. Seventy-two hours later, supernatant was collected, and IFN- y was evaluated by electrochemiluminescence detection utilizing Meso Scale Discovery's human IFN-γ Tissue Culture kit. A schematic of the in vivo and ex vivo elements of the described experiment is shown in FIG. 12A. The fold change in IFN-γ was determined by comparing readouts from ex vivo drug addition to the isotype control group. Tumors taken from six anti-PD-1 treated mice were evaluated. (FIG. 12B). A representative IFN- y plot from one anti-PD-1 pre-exposed tumor stimulated with ex vivo drug treatment is shown in FIG. 12C. ***, p<0.001; **, p<0.01; *, p<0.05.

The data shown in FIGS. 12A-12C indicate that AZD7789 can increase IFN-γ secretion of ex vivo stimulated TILs taken from mice which progressed on anti-PD-1 treatment. This example demonstrates that AZD7789 can improve anti-tumor responses of cells no longer responding to anti-PD-1 therapy.

### 6.11 Example 11

### Sequential Treatment of AZD7789 Following Anti-PD-1 Treatment on Tumor Growth in a Humanized Mouse Tumor Model

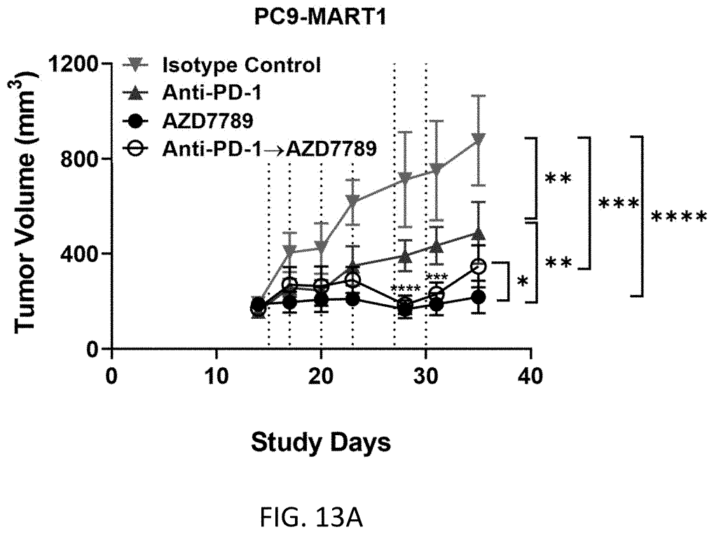

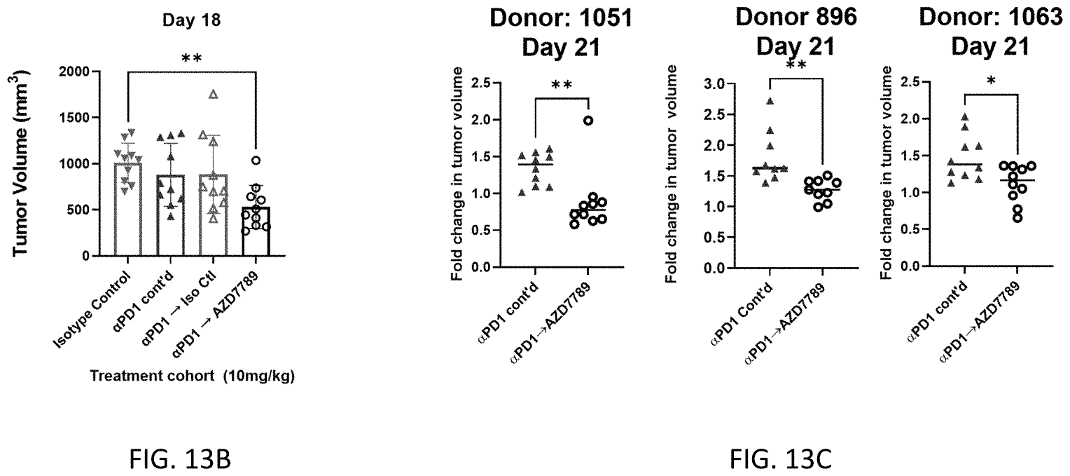

Thirty-two immunodeficient NSG mice were subcutaneously engrafted with 3×10⁶ PC9-MART-1 cells, a human adenocarcinoma cell line engineered to express the melanoma tumor antigen, MART-1. On Day 14, MART-1 reactive CD8+ T cells originating from healthy donor PBMC were intravenously administered (5×10⁶ cells/mouse). Mice were randomized by tumor volume and test and control articles at 10 mg/kg were intraperitoneally administered on Days 15, 17, 20 and then 23, 27 and 30. Mice treated with anti-PD-1 were re-randomized 24 hours after the third dose on Day 21 based on fold change in tumor volume from baseline and were split into 2 cohorts; 10 mice which continued treatment with anti-PD-1, and 10 mice that switched treatment to AZD7789. The tumor volume graph (FIG. 13A) shows treatment comparisons between isotype control, AZD7789, anti-PD-1 mAb LO115 alone, and anti-PD-1 followed by sequential treatment of AZD7789 (three doses of anti-PD-1, followed by three doses of AZD7789); n=8 mice/treatment and all treatments were dosed at 10 mg/kg. Statistics were evaluated by a two-way ANOVA with Tukey's multiple comparisons test. Statistics shown within the graph at time points 5 (Day 28) and 6 (Day 31) compare the anti-PD-1 treatment group to anti-PD-1→AZD7789 treatment group, ****, p<0.0001; ***, p<0.001. All other statistics compare groups at the Day 35 time point, 5 days post last treatment, ****, p<0.0001; ***, p<0.001 **, p<0.01; *, p<0.05.

These results demonstrate that in an antigen-specific humanized mouse tumor model, sequential treatment of AZD7789 following anti-PD-1 treatment can delay tumor growth compared to mice continuously treated with anti-PD-1. This suggests that treatment with AZD7789 may benefit patients that no longer respond to anti-PD-1 therapy.

### 6.12 Example 12

### Effect of Sequential Treatment of AZD7789 Following Anti-PD-1 Treatment on Tumor Growth in a Humanized Mouse Tumor Model

Immunodeficient NSG mice were subcutaneously engrafted with 2×10⁶ OE21-10xGSV3 cells, a human oesophageal squamous cell carcinoma engineered to express viral peptides of interest, on Day 1. Seven days later, viral reactive CD8+ T cells originating from human PBMC isolated from a healthy donor were intravenously administered (1×10⁶ cells/mouse). Mice were randomized by tumor volume on Day 8 into assigned treatment groups. Test and control articles were intraperitoneally administered at 10 mg/kg starting on day 9. In FIG. 13B, mice received 2 doses of isotype control or anti-PD-1 on Days 9 and 11 after which, mice treated with anti-PD-1 were randomized based on fold change in tumor volume from baseline into 3 separate treatment groups, and were subsequently dosed with two doses of either anti-PD-1 (αPD-1 cont'd), isotype control (αPD→Iso Ctl) or AZD7789 (αPD1→AZD7789) on Days 14 and 17. The graph in FIG. 13B depicts the difference in tumor volume between treatment groups at Day 18, 24 hours after the second dose of sequential treatment. Intergroup differences were analyzed for statistical significance by a one-way ANOVA, Tukeys multiple comparison test. In FIG. 13C, mice were treated with anti-PD-1 for 3 doses on Days 9, 13 and 16 prior to randomization on Day 16 into subsequent treatment groups. Studies utilized human PBMC from three healthy donors (D896, D1051, D1063). Each symbol represents the fold-change in tumor volume from the time of re-randomization (post 3 doses of anti-PD-1; Day 16 to 24 hours after the first sequential dose on Day 20. A comparison between the fold-change in tumor volume across the treatment groups was analyzed for statistical significance by an unpaired t-test. The horizontal bars represent the intragroup arithmetic mean fold-change. **, p<0.01; *, p<0.05. n=10 mice per treatment group. All treatments were administered at 10 mg/kg.

This example demonstrates that in a second antigen specific humanized mouse tumor model, sequential treatment of AZD7789 following anti-PD-1 treatment can delay tumor growth, as compared to mice continuously treated with an anti-PD-1 antibody. This result suggests that treatment with AZD7789 may benefit patients no longer responding to anti-PD-1 therapy.

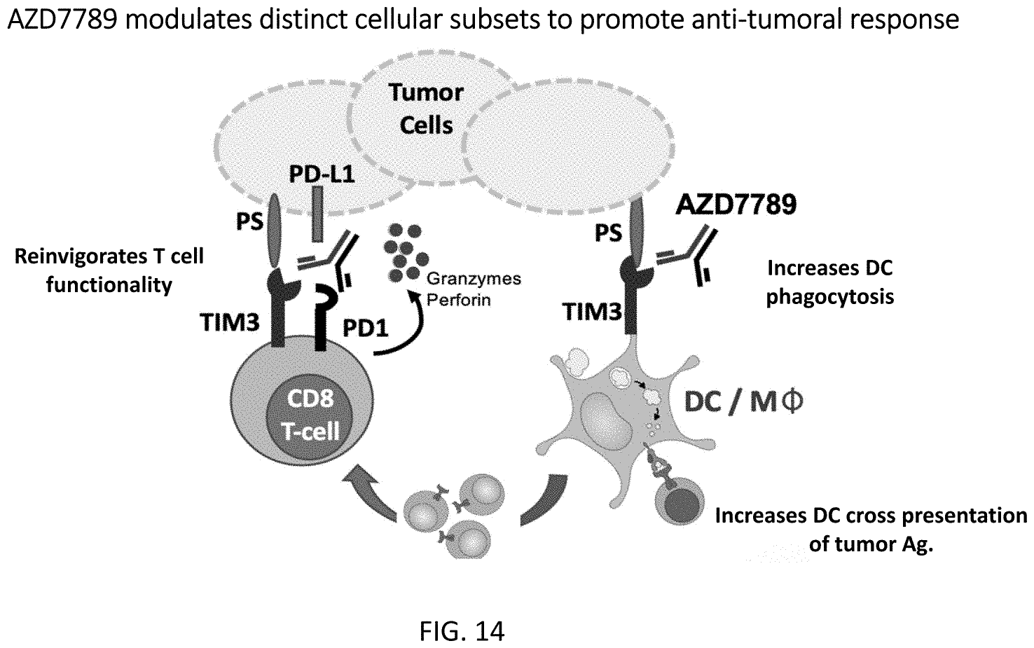

Overall, these examples demonstrate that AZD7789 modulates distinct cellular subsets to promote anti-tumoral response (FIG. 14).
6.13 Example 13: Comparative Characterization of Binding Epitopes

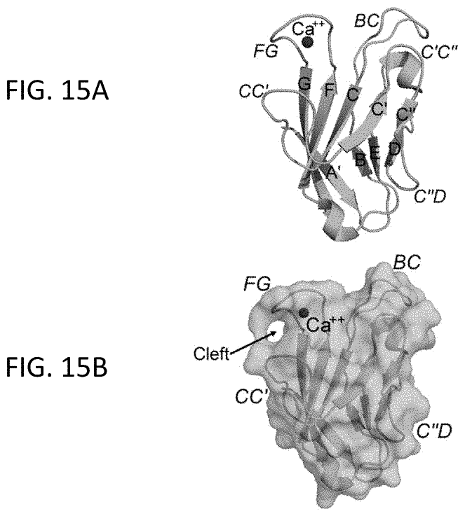

The putative binding epitopes of parent antibody clones O13-1 (the parental clone to AZD7789) and F9S were characterized via various methods and compared to known anti-TIM-3 antibodies. As shown below in Table 5, x-ray crystallography studies and competition binding assays confirmed that mAb O13-1 binds to the C′C″ and DE loops of the TIM-3 IgV domain. By contrast, the majority of the other anti-TIM-3 antibodies tested bound primarily to the CC′ and FG loops (FIGS. 15A and B; see Gandhi et al., Scientific Reports 2018; 8:17512). One tested mAb bound to BC and CC′ loops (WO 2015/117002) and one mAb bound to the DE loops (WO 2016/111947).

As shown in Table 5, methods defined in the table were used to characterize binding to loops of human TIM-3 immunoglobulin variable (IgV) domain bound by anti-TIM-3 antibodies. Each of the references antibodies bound strongly to the listed binding loops, with two exceptions: various antibodies disclosed in WO 2015/117002 bound weakly to the BC loop, and mAb15 disclosed in WO 2016/111947 A2 bound weakly to the DE loop. The antibodies (or derivatives) that bound to the CC′ and FG domains and blocked phosphatidylserine had the strongest reported functional activity as compared to antibodies that bound to the C′C″ and DE loops (WO 2016/111947 A2, US 2018/0016336 A1); antibodies that bound C′C″ and DE loops (WO 2016/071448) were not selected for the most characterized subsequent PD1/TIM3 bispecific antibody (WO 2017/055404).

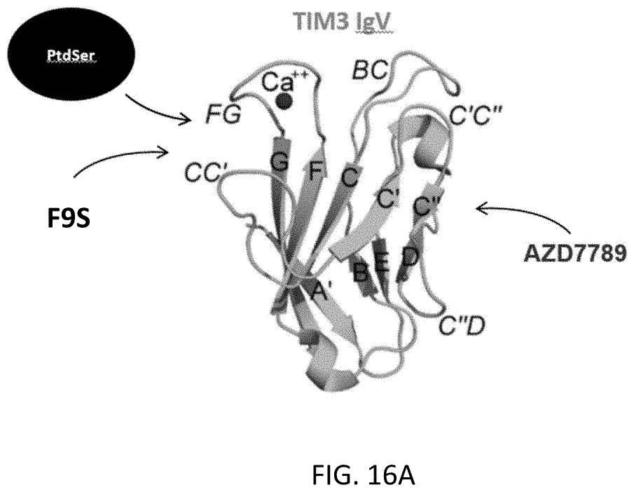

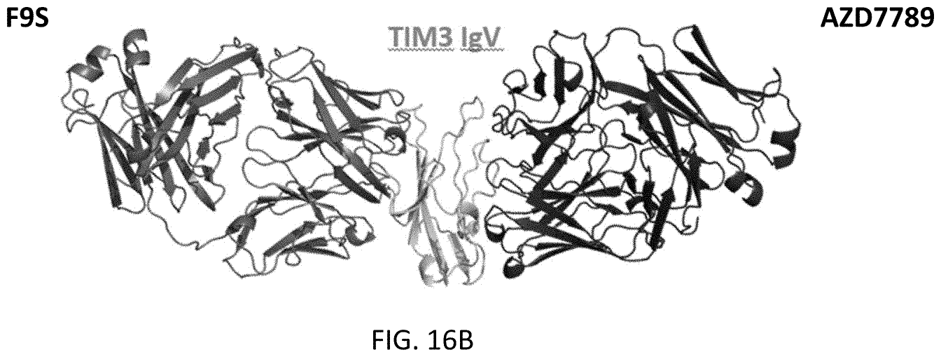

Additionally, as shown in FIGS. 16A and 16B, two in-house developed antibodies, Clone 62 (the TIM-3 arm of AZD7789) and F9S, bind the IgV domain of TIM-3 in non-competitive way. F9S (shown in light grey ribbon in FIG. 16B) binds the IgV domain near the CC′ and FG loops, close to the phosphatidylserine and Ca++ ion binding sites (FIG. 16A). Clone 62 (shown in black cartoon) binds the other side of the IgV beta sandwich. The Clone 62 epitope includes loops BC, C′C″, DE, and short strand D.

This example confirms that AZD7789 binds to a unique epitope on the TIM-3 IgV domain, on the side opposite to phosphatidylserine binding (FIGS. 15A and 15B (Gandhi et al., Scientific Reports 2018; 8:17512)). This binding does not introduce changes into the fold or structure of the IgV domain of TIM-3 and does not block the interaction of TIM-3 with phosphatidylserine (FIG. 2). Instead, AZD7789 increases engagement between TIM-3 and phosphatidylserine (FIG. 2). This unique mechanism improves T cell mediated anti-tumor responses over those observed from known phosphatidylserine blocking anti-TIM3 antibodies (FIGS. 11-13).

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

TABLE of sequences

| Description | Sequence |
|---|---|
| AZD 7789 | SYAMS |
| TIM-3 | (SEQ ID NO: 1) |
| VH CDR1 | |
| AZD 7789 | AISGSGGSTYYADSVKG |
| TIM-3 | (SEQ ID NO: 2) |
| VH CDR2 | |
| AZD 7789 | GSYGTYYGNYFEY |
| TIM-3 | (SEQ ID NO: 3) |
| VH CDR3 | |
| AZD 7789 | DYGMH |
| PD-1 | (SEQ ID NO: 4) |
| VH CDR1 | |
| AZD 7789 | YISSGSYTIYSADSVKG |
| PD-1 | (SEQ ID NO: 5) |
| VH CDR2 | |
| AZD 7789 | RAPNSFYEYYFDY |
| PD-1 | (SEQ ID NO: 6) |
| VH CDR3 | |
| AZD 7789 | GGDNIGGKSVH |
| TIM-3 | (SEQ ID NO: 7) |
| VL CDR1 | |
| AZD 7789 | YDSDRPS |
| TIM-3 | (SEQ ID NO: 8) |
| VL CDR2 | |
| AZD 7789 | QVLDRRSDHFL |
| TIM-3 | (SEQ ID NO: 9) |
| VL CDR3 | |
| AZD 7789 | SASSKHTNLYWSRHMYWY |
| PD-1 | (SEQ ID NO: 10) |
| VL CDR1 | |
| AZD 7789 | LTSNRAT |
| PD-1 | (SEQ ID NO: 11) |
| VL CDR2 | |
| AZD 7789 | QQWSSNP |
| PD-1 | (SEQ ID NO: 12) |
| VL CDR3 | |
| TIM-3 (#62) | QVLDRRSDHWL |
| VL CDR3 | (SEQ ID NO: 13) |
| AZD 7789 | EVQLLESGGGLVQPGGSLRLSCAASGFTFSSYAMSWVRQAPGK |
| TIM-3 | GLEWVSAISGSGGSTYYADSVKGRFTISRDNSKNTLYLQMNSLR |
| VH | AEDTAVYYCARGSYGTYYGNYFEYWGQGTLVTVSS |
| | (SEQ ID NO: 14) |
| AZD 7789 | EVQLLESGGGLVQPGGSLRLSCAASGFTFSSYAMSWVRQAPGK |
| TIM-3 | GLEWVSAISGSGGSTYYADSVKGRFTISRDNSKNTLYLQMNSLR |
| HC | AEDTAVYYCARGSYGTYYGNYFEYWGQGTLVTVSSASTKGPS |
| | VCPLAPSSKSTSGGTAALGCLVKDYFPEPVTVSWNSGALTSGVH |
| | TFPAVLQSSGLYSLSSVVTVPSSSLGTQTYICNVNHKPSNTKVDK |
| | RVEPKSVDKTHTCPPCPAPEFEGGPSVFLFPPKPKDTLMISRTPEV |
| | TCVVVDVSHEDPEVKFNWYVDGVEVHNAKTKPREEQYNSTYR |
| | VVSVLTVLHQDWLNGKEYKCKVSNKALPASIEKTISKAKGQPR |
| | EPQVYTLPPCREEMTKNQVSLWCLVKGFYPSDIAVEWESNGQP |
| | ENNYKTTPPVLDSDGSFFLYSKLTVDKSRWQQGNVFSCSVMHE |
| | ALHNHYTQKSLSLSPGK |
| | (SEQ ID NO: 15) |
| TIM3 (#62) | QTVLTQPPSVSVAPGKTASISCGGDNIGGKSVHWYQQKPGQAP |
| Variable | VLVIYYDSDRPSGIPQRFSGSNSGNTATLTIHRVEAGDEADYYCQ |
| Light VL | VLDRRSDHWLFGGGTKLTVL (SEQ ID NO: 16) |
| AZD 7789 | SYVLTQPPSVSVAPGKTARITCGGDNIGGKSVHWYQQKPGQAP |
| TIM-3 | VLVIYYDSDRPSGIPERFSGSNSGNTATLTISRVEAGDEADYYCQ |
| VL | VLDRRSDHFLFGGGTKLTVL |
| | (SEQ ID NO: 17) |
| AZD 7789 | SYVLTQPPSVSVAPGKTARITCGGDNIGGKSVHWYQQKPGQAP |
| TIM-3 | VLVIYYDSDRPSGIPERFSGSNSGNTATLTISRVEAGDEADYYCQ |
| LC | VLDRRSDHFLFGGGTKLTVLGQPKAAPSVTLFPPCSEELQANKA |
| | TLVCLISDFYPGAVTVAWKADSSPVKAGVETTTPSKQSNNKYA |
| | ASSYLSLTPEQWKSHRSYSCQVTHEGSTVEKTVAPTEVS |
| | (SEQ ID NO: 18) |
| AZD | EVQLVESGGGLVQPGGSLRLSCAASGFTFSDYGMHWVRQAPGK |
| 7789 | GLEWVAYISSGSYTIYSADSVKGRFTISRDNAKNSLYLQMNSLR |
| PD-1 | AEDTAVYYCARRAPNSFYEYYFDYWGQGTTVTVSS |
| VH | (SEQ ID NO: 19) |
| AZD 7789 | EVQLVESGGGLVQPGGSLRLSCAASGFTFSDYGMHWVRQAPGK |
| PD-1 | GLEWVAYISSGSYTIYSADSVKGRFTISRDNAKNSLYLQMNSLR |
| HC | AEDTAVYYCARRAPNSFYEYYFDYWGQGTTVTVSSASTKGPSV |
| | FPLAPSSKSTSGGTAALGCLVKDYFPEPVTVSWNSGALTSGVHT |
| | FPAVLQSSGLYSLSSVVTVPSSSLGTQTYICNVNHKPSNTKVDKR |
| | VEPKSCDKTHTCPPCPAPEFEGGPSVFLFPPKPKDTLMISRTPEVT |
| | CVVVDVSHEDPEVKFNWYVDGVEVHNAKTKPREEQYNSTYRV |
| | VSVLTVLHQDWLNGKEYKCKVSNKALPASIEKTISKAKGQPREP |
| | QVCTLPPSREEMTKNQVSLSCAVKGFYPSDIAVEWESNGQPENN |
| | YKTTPPVLDSDGSFFLVSKLTVDKSRWQQGNVFSCSVMHEALH |
| | NHYTQKSLSLSPGK |
| | (SEQ ID NO: 20) |
| AZD 7789 | QIVLTQSPATLSLSPGERATLSCSASSKHTNLYWSRHMYWYQQ |
| PD-1 | KPGQAPRLLIYLTSNRATGIPARFSGSGSGTDFTLTISSLEPEDFA |
| VL | VYYCQQWSSNPFTFGQGTKLEIK |
| | (SEQ ID NO: 21) |
| AZD 7789 | QIVLTQSPATLSLSPGERATLSCSASSKHTNLYWSRHMYWYQQ |
| PD-1 | KPGQAPRLLIYLTSNRATGIPARFSGSGSGTDFTLTISSLEPEDFA |
| LC | VYYCQQWSSNPFTFGQGTKLEIKRTVAAPSVFIFPPSDEQLKSGT |
| | ASVVCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 22) |
| TIM-3 Heavy | EVQLVESGGGLVQPGGSLRLSCAASGFTFSDYGMHWVRQAPGK |
| Chain | GLEWVAYISSGSYTIYSADSVKGRFTISRDNAKNSLYLQMNSLR |
| | AEDTAVYYCARRAPNSFYEYYFDYWGQGTTVTVSSASTKGPSV |
| | FPLAPSSKSTSGGTAALGCLVKDYFPEPVTVSWNSGALTSGVHT |
| | FPAVLQSSGLYSLSSVVTVPSSSLGTQTYICNVNHKPSNTKVDKR |
| | VEPKSCDKTHTCPPCPAPEFEGGPSVFLFPPKPKDTLMISRTPEVT |
| | CVWDVSHEDPEVKFNWYVDGVEVHNAKTKPREEQYNSTYRW |
| | SVLTVLHQDWLNGKEYKCKVSNKALPASIEKTISKAKGQPREPQ |
| | VYTLPPSREEMTKNQVSLTCLVKGFYPSDIAVEWESNGQPENNY |
| | KTTPPVLDSDGSFFLYSKLTVDKSRWQQGNVFSCSVMHEALHN |
| | HYTQKSLSLSPGKGGGGSGGGGSEVQLLESGGGLVQPGGSLRLS |
| | CAASGFTFSSYAMSWVRQAPGKCLEWVSAISGSGGSTYYADS |
| | VKGRFTISRDNSKNTLYLQMNSLRAEDTAVYYCARGSYGTYYG |
| | NYFEYWGQGTLVTVSSGGGGSGGGGSGGGGSGGGGSSYVLTQ |
| | PPSVSVAPGKTARITCGGDNIGGKSVHWYQQKPGQAPVLVIYY |
| | DSDRPSGIPERFSGSNSGNTATLTISRVEAGDEADYYCQVLDRRS |
| | DHFLFGCGTKLTVL |
| | (SEQ ID NO: 23) |
| TIM-3 Light Chain | QIVLTQSPATLSLSPGERATLSCSASSKHTNLYWSRHMYWYQQ |
| Variable Region | KPGQAPRLLIYLTSNRATGIPARFSGSGSGTDFTLTISSLEPEDFA |
| | VYYCQQWSSNPFTFGQGTKLEIKRTVAAPSVFIFPPSDEQLKSGT |
| | ASWCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 24) |
| TIM-3 Heavy Chain | EVQLVESGGGLVQPGGSLRLSCAASGFTFSDYGMHWVRQAPGK |
| | GLEWVAYISSGSYTIYSADSVKGRFTISRDNAKNSLYLQMNSLR |
| | AEDTAVYYCARRAPNSFYEYYFDYWGQGTTVTVSSASTKGPSV |
| | FPLAPSSKSTSGGTAALGCLVKDYFPEPVTVSWNSGALTSGVHT |
| | FPAVLQSSGLYSLSSVVTVPSSSLGTQTYICNVNHKPSNTKVDKR |
| | VEPKSCDKTHTCPPCPAPEFEGGPSVFLFPPKPKDTLMISRTPEVT |
| | CVWDVSHEDPEVKFNWYVDGVEVHNAKTKPREEQYNSTYRW |
| | SVLTVLHQDWLNGKEYKCKVSNKALPASIEKTISKAKGQPREPQ |
| | VYTLPPSREEMTKNQVSLTCLVKGFYPSDIAVEWESNGGGGSG |
| | GGGSEVQLLESGGGLVQPGGSLRLSCAASGFTFSSYAMSWVRQ |
| | APGKCLEWVSAISGSGGSTYYADSVKGRFTISRDNSKNTLYLQM |
| | NSLRAEDTAVYYCARGSYGTYYGNYFEYWGQGTLVTVSSGGG |
| | GSGGGGSGGGGSGGGGSSYVLTQPPSVSVAPGKTARITCGGDNI |
| | GGKSVHWYQQKPGQAPVLVIYYDSDRPSGIPERFSGSNSGNTAT |
| | LTISRVEAGDEADYYCQVLDRRSDHFLFGCGTKLTVLGGGGSG |
| | GGGSGQPENNYKTTPPVLDSDGSFFLYSKLTVDKSRWQQGNVF |
| | SCSVMHEALHNHYTQKSLSLSPGK |
| | (SEQ ID NO: 25) |
| TIM-3 Light Chain | QIVLTQSPATLSLSPGERATLSCSASSKHTNLYWSRHMYWYQQ |
| | KPGQAPRLLIYLTSNRATGIPARFSGSGSGTDFTLTISSLEPEDFA |
| | VYYCQQWSSNPFTFGQGTKLEIKRTVAAPSVFIFPPSDEQLKSGT |
| | ASWCLLNNFYPREAKVQWKVDNALQSGNSQESVTEQDSKDST |
| | YSLSSTLTLSKADYEKHKVYACEVTHQGLSSPVTKSFNRGEC |
| | (SEQ ID NO: 26) |
| TIM3 (#62) Variable | EVQLLESGGGLVQPGGSLRLSCAASGFTFSSYAMSWVRQAPGK |
| Heavy VH | GLEWVSAISGSGGSTYYADSVKGRFTISRDNSKNTLYLQMNSLR |
| | AEDTAVYYCARGSYGTYYGNYFEYWGRGTLVTVSS (SEQ ID |
| | NO: 27) |
| Amino acid sequence | MQIPQAPWPVVWAVLQLGWRPGWFLDSPDRPWNPPTFSPALLV |
| of human PD-1 | VTEGDNATFTCSFSNTSESFVLNWYRMSPSNQTDKLAAFPEDRS |
| protein | QPGQDCRFRVTQLPNGRDFHMSVVRARRNDSGTYLCGAISLAP |
| | KAQIKESLRAELRVTERRAEVPTAHPSPSPRPAGQFQTLVVGVV |
| | GGLLGSLVLLVWVLAVICSRAARGTIGARRTGQPLKEDPSAVPV |
| | FSVDYGELDFQWREKTPEPPVPCVPEQTEYATIVFPSGMGTSSPA |
| | RRGSADGPRSAQPLRPEDGHCSWPL |
| | (SEQ ID NO: 28) |
| Human TIM-3 IgV | SEVEYRAEVGQNAYLPCFYTPAAPGNLVPVCWGKGACPVFECG |
| domain | NVVLRTDERDVNYWTSRYWLNGDFRKGDVSLTIENVTLADSGI |
| | YCCRIQIPGIMNDEKFNLKLVIK |
| | (SEQ ID NO: 29) |
| Human TIM-3 protein | MFSHLPFDCVLLLLLLLLTRSSEVEYRAEVGQNAYLPCFYTPAA |
| | PGNLVPVCWGKGACPVFECGNVVLRTDERDVNYWTSRYWLN |
| | GDFRKGDVSLTIENVTLADSGIYCCRIQIPGIMNDEKFNLKLVIKP |
| | AKVTPAPTRQRDFTAAFPRMLTTRGHGPAETQTLGSLPDINLTQI |
| | STLANELRDSRLANDLRDSGATIRIGIYIGAGICAGLALALIFGAL |
| | IFKWYSHSKEKIQNLSLISLANLPPSGLANAVAEGIRSEENIYTIEE |
| | NVYEVEEPNEYYCYVSSRQQPSQPLGCRFAMP |
| | (SEQ ID NO: 30) |

1. A method of altering engagement between T-cell immunoglobulin and mucin domain containing protein-3 (TIM-3) and phosphatidylserine (PS) in a subject, the method comprising administering to the subject a TIM-3 binding protein comprising a TIM-3 binding domain, wherein the TIM-3 binding domain specifically binds to the C′C″ and DE loops of the immunoglobulin variable (IgV) domain of TIM-3.

2. The method of claim 1, wherein administration of the TIM-3 binding protein increases anti-tumor activity in a subject relative to no antibody administration.

3. The method of claim 1, wherein administration of the TIM-3 binding protein increases anti-tumor activity in a subject relative to administration of a TIM-3 binding protein that binds to the PS binding cleft (FG and CC′ loops) of the IgV domain of TIM-3.

4. A method of increasing T cell mediated anti-tumor activity in a subject, the method comprising administering to the subject a TIM-3 binding protein comprising TIM-3 binding domain, wherein the TIM-3 binding domain specifically binds to the C′C″ and DE loops of the IgV domain of TIM-3.

5. The method of claim 4, wherein the T cell mediated anti-tumor activity in the subject is increased relative to no antibody administration.

6. The method of claim 4, wherein the T cell mediated anti-tumor activity in the subject is increased relative to administration of a TIM-3 binding protein that binds to the PS binding cleft (FG and CC′ loops) of the IgV domain of TIM-3.

7. The method of claim 1, wherein administration of the TIM-3 binding protein increases dendritic cell phagocytosis of apoptotic tumor cells in a subject relative to no antibody administration.

8. The method of claim 1, wherein administration of the TIM-3 binding protein increases dendritic cell phagocytosis of apoptotic tumor cells in a subject relative to administration of a TIM-3 binding protein that binds to the PS binding cleft (FG and CC′ loops) of the IgV domain of TIM-3.

9. The method of claim 1, wherein administration of the TIM-3 binding protein increases dendritic cell cross-presentation of tumoral antigen in a subject relative to no antibody administration.

10. The method of claim 1, wherein administration of the TIM-3 binding protein increases dendritic cell cross-presentation of tumoral antigen in a subject relative to administration of a TIM-3 binding protein that binds to the PS binding cleft (FG and CC′ loops) of the IgV domain of TIM-3.

11. A method of promoting dendritic cell phagocytosis of tumor cells in a subject, the method comprising administering to the subject a TIM-3 binding protein comprising a TIM-3 binding domain, wherein the TIM-3 binding domain specifically binds to the C′C″ and DE loops of the IgV domain of TIM-3.

12. A method of increasing dendritic cell cross-presentation of tumor antigens in a subject, the method comprising administering to the subject a TIM-3 binding protein comprising a TIM-3 binding domain, wherein the TIM-3 binding domain specifically binds to the C′C″ and DE loops of the IgV domain of TIM-3.

13. The method of claim 12, wherein the level of dendritic cell cross-presentation is increased relative to no antibody administration.

14. The method of claim 12, wherein the level of dendritic cell cross-presentation is increased relative to administration of a TIM-3 binding protein that binds to the PS binding cleft (FG and CC′ loops) of the IgV domain of TIM-3.

15. The method of claim 1, wherein administration of the TIM-3 binding protein increases IL-2 secretion upon engagement to TIM-3 positive T cells in a subject relative to no antibody administration.

16. The method of claim 1, wherein administration of the TIM-3 binding protein increases IL-2 secretion upon engagement to TIM-3 positive T cells in a subject relative to administration of a TIM-3 binding protein that binds to the PS binding cleft (FG and CC′ loops) of the IgV domain of TIM-3.

17. The method of claim 1, wherein administration of the TIM-3 binding protein results in inhibition of tumor growth in the subject.

18. The method of claim 17, wherein the tumor is an advanced or metastatic solid tumor.

19. The method of claim 1, wherein the subject has one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, lung cancer, esophageal cancer, gastric cancer, biliary tract tumors, urothelial carcinoma, Hodgkin lymphoma, non-hodgkin lymphoma, myelodysplastic syndrome, and acute myeloid leukemia.

20. The method of claim 1, wherein the subject has immuno-oncology (IO) acquired resistance.

21. A method of treating a cancer in a subject with IO acquired resistance, wherein the method comprises administering to the subject a TIM-3 binding protein comprising a TIM-3 binding domain, wherein the TIM-3 binding domain specifically binds to the C′C″ and DE loops of the IgV domain of TIM-3.

22. The method of claim 21, wherein the cancer is one or more of ovarian cancer, breast cancer, colorectal cancer, prostate cancer, cervical cancer, uterine cancer, testicular cancer, bladder cancer, head and neck cancer, melanoma, pancreatic cancer, renal cell carcinoma, lung cancer, esophageal cancer, gastric cancer, biliary tract tumors, urothelial carcinoma, Hodgkin lymphoma, non-hodgkin lymphoma, myelodysplastic syndrome, and acute myeloid leukemia.

23. The method of claim 1, wherein the subject is a human.

24. The method of claim 1, wherein the subject has documented Stage III which is not amenable to curative surgery or radiation, or Stage IV non-small cell lung carcinoma (NSCLC).

25. The method of claim 24, wherein the NSCLC is squamous or non-squamous NSCLC.

26. The method of claim 1, wherein the subject has a radiologically documented tumor progression or clinical deterioration following initial treatment with an anti-PD-1/PD-L1 therapy for a minimum of 3-6 months, as monotherapy or in combination with chemotherapy, and had signs of initial clinical benefit, i.e. disease stabilization or regression.

27. The method of claim 20, wherein the IO acquired resistance is defined as:
(i) Exposure of less than 6 months to anti-PD-1/PD-L1 monotherapy with initial best overall response (BOR) of partial regression or complete regression followed by disease progression during treatment or disease progression less than or equal to 12 weeks after anti-PD-1/PD-L1 treatment discontinuation; or
(ii) Exposure of greater than or equal to 6 months to anti-PD-1/PD-L1 therapy alone or in combination with chemotherapy with BOR of disease stabilization, partial regression, or complete regression followed by disease progression during treatment or disease progression less than or equal to 12 weeks after anti-PD-1/PD-L1 treatment discontinuation.

28. The method of claim 20, wherein the IO acquired resistance is defined as exposure of greater than or equal to 6 months to anti-PD-1/PD-L1 therapy alone or in combination with chemotherapy; a best overall response (BOR) of disease stabilization, partial regression, or complete regression followed by disease progression during treatment or disease progression less than or equal to 12 weeks after anti-PD-1/PD-L1 treatment discontinuation.

29. The method of claim 1, wherein the subject's PD-L1 tumor proportion score (TPS) is greater than or equal to 1%.

30. The method of claim 1, wherein the subject has not received prior systemic therapy in a first-line setting.

31. The method of 30, wherein the prior systemic therapy is an IO therapy other than an anti-PD-1/PD-L1 therapy.

32. The method of claim 30, wherein the subject received prior neo/adjuvant therapy but did not progress for at least 12 months following the last administration of an anti-PD-1/PD-L1 therapy.

33. The method of claim 32, wherein the subject's PD-L1 TPS is greater than or equal to 50%.

34. The method of claim 1, wherein the TIM-3 binding protein comprises Complementarity-Determining Regions (CDRs): HCDR1, HCDR2, HCDR3, LCDR1, LCDR2, and LCDR3 comprising the amino acid sequences of SEQ ID NOs: 1, 2, 3, 7. 8, and 9, respectively, or SEQ ID NOs: 1, 2, 3, 7, 8, and 13, respectively.

35. The method of claim 1, wherein the TIM-3 binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 29).

36. The method of claim 1, wherein the TIM-3 binding protein further comprises a Programmed cell death protein 1 (PD-1) binding domain.

37. The method of claim 36, the TIM-3 binding domain comprises a first set of Complementarity-Determining Regions (CDRs): HCDR1, HCDR2, HCDR3, LCDR1, LCDR2, and LCDR3 comprising the amino acid sequences of SEQ ID NOs: 1, 2, 3, 7, 8, and 9 or 1, 2, 3, 7, 8, and 13, respectively; and
the PD-1 binding domain comprises a second set of CDRs: HCDR1, HCDR2, HCDR3, LCDR1, LCDR2, and LCDR3 comprising the amino acid sequences of SEQ ID NOs: 4, 5, 6, 10, 11, and 12, respectively.

38. The method of claim 37, wherein the TIM-3 binding protein comprises a first heavy chain variable domain (VH) comprising the amino acid sequence of SEQ ID NO: 14, a first light chain variable domain (VL) comprising the amino acid sequence of SEQ ID NO: 17, a second heavy chain VH comprising the amino acid sequence of SEQ ID NO: 19, and a second light chain VL comprising the amino acid sequence of SEQ ID NO: 21.

39. The method of claim 37, wherein the TIM-3 binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22.

40. The method of claim 37, wherein the TIM-3 binding protein comprises a first heavy chain comprising the amino acid sequence of SEQ ID NO: 23, a first light chain comprising the amino acid sequence of SEQ ID NO: 24, a second heavy chain comprising the amino acid sequence of SEQ ID NO: 23, and a second light chain comprising the amino acid sequence of SEQ ID NO: 24.

41. The method of claim 37, wherein the TIM-3 binding protein comprises a first heavy chain comprising the amino acid sequence of SEQ ID NO: 25, a first light chain comprising the amino acid sequence of SEQ ID NO: 26, a second heavy chain comprising the amino acid sequence of SEQ ID NO: 25, and a second light chain comprising the amino acid sequence of SEQ ID NO: 26.

42. The method of claim 24, wherein the TIM-3 binding protein comprises an aglycosylated Fc region.

43. The method of claim 24, wherein the TIM-3 binding protein comprises a deglycosylated Fc region.

44. The method of claim 24, wherein the TIM-3 binding protein comprises an Fc region which has reduced fucosylation or is afucosylated.

45. A method of treating NSCLC in a subject having advanced or metastatic NSCLC, the method comprising administering to the subject a bispecific binding protein comprising a PD-1 binding domain and a TIM-3 binding domain,
wherein the bispecific binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22,
and wherein the subject has IO acquired resistance.

46. A method of inhibiting growth of a non-small cell lung tumor in a subject having an advanced or metastatic tumor, the method comprising administering to the subject a bispecific binding protein comprising a PD-1 binding domain and a TIM-3 binding domain,
wherein the bispecific binding protein comprises a first heavy chain comprising the amino acid sequence of SEQ ID NO: 15, a first light chain comprising the amino acid sequence of SEQ ID NO: 18, a second heavy chain comprising the amino acid sequence of SEQ ID NO: 20, and a second light chain comprising the amino acid sequence of SEQ ID NO: 22,
and wherein the subject has IO acquired resistance.

47. The method of claim 45, wherein the TIM-3 binding domain specifically binds to the C′C″ and DE loops of the IgV domain of TIM-3.

48. The method of claim 45, wherein the TIM-3 binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 29).

49. The method of any one of claims claim 45-48, wherein the NSCLC is squamous or non-squamous NSCLC.

50. The method of claim 46, wherein the TIM-3 binding domain specifically binds to the C′C″ and DE loops of the IgV domain of TIM-3.

51. The method of claim 46, wherein the TIM-3 binding domain specifically binds to epitopes on the IgV domain of TIM-3 and the epitopes comprises N12, L47, R52, D53, V54, N55, Y56, W57, W62, L63, N64, G65, D66, F67, R68, K69, D71, T75, and E77 of TIM-3 (SEQ ID NO: 29).

52. The method of claim 46, wherein the NSCLC is squamous or non-squamous NSCLC.

53. The method of claim 45, wherein the subject has a radiologically documented tumor progression or clinical deterioration following initial treatment with an anti-PD-1/PD-L1 therapy for a minimum of 3-6 months, as monotherapy or in combination with chemotherapy, and had signs of initial clinical benefit, i.e. disease stabilization or regression.

54. The method of claim 45, wherein the IO acquired resistance is defined as:
(i) Exposure of less than 6 months to anti-PD-1/PD-L1 monotherapy with initial best overall response (BOR) of partial regression or complete regression followed by disease progression during treatment or disease progression less than or equal to 12 weeks after anti-PD-1/PD-L1 treatment discontinuation; or
(ii) Exposure of greater than or equal to 6 months to anti-PD-1/PD-L1 therapy alone or in combination with chemotherapy with BOR of disease stabilization, partial regression, or complete regression followed by disease progression during treatment or disease progression less than or equal to 12 weeks after anti-PD-1/PD-L1 treatment discontinuation.

55. The method of claim 45, wherein the IO acquired resistance is defined as exposure of greater than or equal to 6 months to anti-PD-1/PD-L1 therapy alone or in combination with chemotherapy; a best overall response (BOR) of disease stabilization, partial regression, or complete regression followed by disease progression during treatment or disease progression less than or equal to 12 weeks after anti-PD-1/PD-L1 treatment discontinuation.

56. The method of claim 46, wherein the subject has a radiologically documented tumor progression or clinical deterioration following initial treatment with an anti-PD-1/PD-L1 therapy for a minimum of 3-6 months, as monotherapy or in combination with chemotherapy, and had signs of initial clinical benefit, i.e. disease stabilization or regression.

57. The method of claim 46, wherein the IO acquired resistance is defined as:
(i) Exposure of less than 6 months to anti-PD-1/PD-L1 monotherapy with initial best overall response (BOR) of partial regression or complete regression followed by disease progression during treatment or disease progression less than or equal to 12 weeks after anti-PD-1/PD-L1 treatment discontinuation; or
(ii) Exposure of greater than or equal to 6 months to anti-PD-1/PD-L1 therapy alone or in combination with chemotherapy with BOR of disease stabilization, partial regression, or complete regression followed by disease progression during treatment or disease progression less than or equal to 12 weeks after anti-PD-1/PD-L1 treatment discontinuation.

58. The method of claim 46, wherein the IO acquired resistance is defined as exposure of greater than or equal to 6 months to anti-PD-1/PD-L1 therapy alone or in combination with chemotherapy; a best overall response (BOR) of disease stabilization, partial regression, or complete regression followed by disease progression during treatment or disease progression less than or equal to 12 weeks after anti-PD-1/PD-L1 treatment discontinuation.
